(19) United States
(12) Patent Application Publication
(10) Pub. No.: US 2025/0268873 A1
(43) Pub. Date: Aug. 28, 2025

(54) COMBINATION OF DUAL ATM AND DNA-PK INHIBITORS AND IMMUNOTHERAPEUTIC AGENTS FOR USE IN CANCER THERAPY

(71) Applicant: XRAD THERAPEUTICS, INC., Orlando, FL (US)

(72) Inventors: Tona GILMER, Orlando, FL (US); Michael KASTAN, Orlando, FL (US); David KIRSCH, Orlando, FL (US)

(73) Assignee: XRAD THERAPEUTICS, INC., Orlando, FL (US)

(21) Appl. No.: 18/263,998

(22) PCT Filed: Feb. 2, 2022
(86) PCT No.: PCT/US2022/014900

§ 371 Date: Aug. 2, 2023

Related U.S. Application Data
(60) Provisional application No. 63/277,672, filed on Nov. 10, 2021.
(60) Provisional application No. 63/145,321, filed on Feb. 3, 2021.

(51) Int. Cl. A61K 31/437; A61K 39/00; A61K 39/395; A61P 35/00; C07D 471/04

(52) U.S. Cl. CPC A61K 31/437; A61K 39/39558; A61P 35/00; C07D 471/04; A61K 2039/505

(57) ABSTRACT

A combined cancer therapy comprising a compound of Formula (I) or a pharmaceutically acceptable salt thereof, an immune checkpoint inhibitor, and optionally a radiotherapy.

### CROSS-REFERENCE TO RELATED APPLICATIONS

This application is a United States National Phase under 35 U.S.C. § 371 of International Application No. PCT/US2022/014900, filed Feb. 2, 2022, which claims the benefit of U.S. Patent Application No. 62/145,321, filed on Feb. 3, 2021, and U.S. Patent Application No. 63/277,672, filed on Nov. 10, 2021, the contents of each of which are incorporated by reference herein in its entirety.

### BACKGROUND OF THE INVENTION

Several members of the PIKK (PI-3K-like Kinase) family of serine-threonine kinases are known mediators of DNA damage signaling.

Radiation therapy (RT) is used to treat >50% of all cancer patients at some point during their illness. Despite significant effort, previous approaches to develop clinical radiosensitizers have not been highly effective, primarily as a result of targeting non-specific pathways which are not direct regulators of the cellular response to radiation.

Immunotherapy refers to treatment of diseases by modulating the immune system. In recent years, immunotherapy has shown positive results in treatment of various forms of cancer. It however can also cause problems given the complexity of the immune system and variations from human to human.

There is a need for new therapies for oncological diseases.

### SUMMARY OF THE INVENTION

The present disclosure is based, at least in part, on the development of selective, dual inhibitors of ATM and DNA-PK kinases and their combined use with immune checkpoint inhibitors in cancer treatment.

In some aspects, the present disclosure features a method for treating cancer, comprising administering to a subject in need thereof an effective amount of a compound of Formula (I):

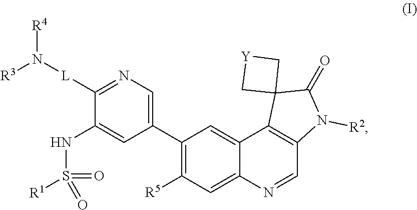

or a pharmaceutically acceptable salt thereof. In Formula (I) R¹, R², R³, R⁴, R⁶, and R⁷ each independently can be H, or C₁₋₃ alkyl
R⁵ can be H or halogen (e.g., flurorine); Y can be CHR⁶ or NR⁷; and/or
L can be —OR⁸—, or —N(R⁸)₂—, in which
each R⁸ can independently be H or C₁₋₃ alkyl, or both R⁸ groups, together with N, form a heterocyclyl ring. In some embodiments, the subject has received or is receiving an anti-tumor immune checkpoint inhibitor.

In other aspects, the present disclosure features a method for treating cancer, comprising administering to a subject in need thereof (a) an effective amount of an anti-tumor immune checkpoint inhibitor, and (b) an effective amount of the compound of Formula (I) or a pharmaceutically acceptable salt thereof.

In any of the methods disclosed herein, the subject has received or is receiving a radiotherapy. Alternatively, the method may further comprise performing a radiotherapy to the subject.

In some embodiments, the compound used in any of the methods disclosed herein can be of Formula (Ia):

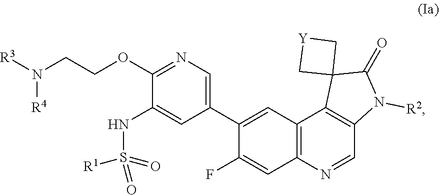

in which Y and R¹-R⁴ are as described herein.

In some examples, the compound of Formula (Ia) is as follows:

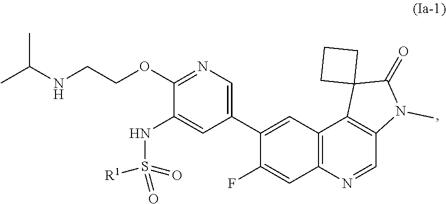

or a pharmaceutically acceptable salt thereof. In some instances, R¹ can be C₁₋₃ alkyl.

In some examples, the compound of Formula (Ia-1) is

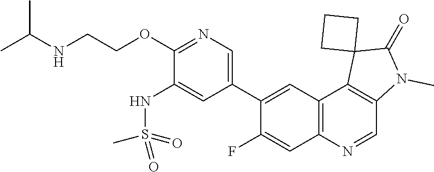 [SMILES: CC(C)NCCOc1ncc(-c2cc3c4c(cnc3cc2F)N(C)C(=O)C42CCC2)cc1NS(C)(=O)=O]

or a pharmaceutically acceptable salt thereof.

In some examples, the compound to be used in any of the methods disclosed herein can be of Formula (Ib):

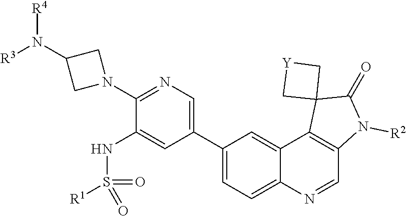

Y and R¹⁻⁴ are as defined herein.

In some examples, a mesylate salt of the compound of Formula (I) can be used in any of the methods disclosed herein.

In some embodiments, the compound of Formula (I) is administered orally. For example, the compound of Formula (I) is administered once or twice a day.

In some embodiments, the checkpoint inhibitor is a PD-1 antagonist, which optionally is selected from the PD-1 antagonists listed in Table 1. In some examples, the PD-1 antagonist is an anti-PD-1 antibody, which optionally is selected from the anti-PD-1 antibodies listed in Table 1. In some examples, the PD-1 antagonist is an anti-PD-L1 antibody, which optionally is selected from the anti-PD-L1 antibodies listed in Table 1. Any of the checkpoint inhibitors disclosed herein may be administered by intravenous infusion. Alternatively, the checkpoint inhibitor may be administered orally.

In any of the methods disclosed herein, the subject can be a human cancer patient. For example, the human cancer patient has colon cancer, melanoma, breast cancer, lung cancer or head and neck cancer (e.g., hypopharyngeal carcinoma).

In other aspects, provided herein is a mesylate salt of a compound of Formula (I):

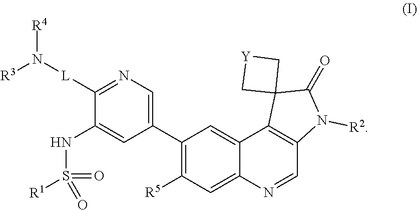

In Formula (I), each of R¹, R², R³, R⁴, R⁶, and R⁷ independently can be H, or C₁₋₃ alkylR⁵ can be H or halogen; Y can be CHR⁶ or NR⁷; and/or
L can be —OR⁸—, or —N(R⁸)₂—, in which each R⁸ can independently be H or C₁₋₃ alkyl. Alternatively or both R⁸ substituents, together with N, form a heterocyclyl ring; and

In some embodiments, the mesylate salt can be a mesylate of the compound is of Formula (Ia):

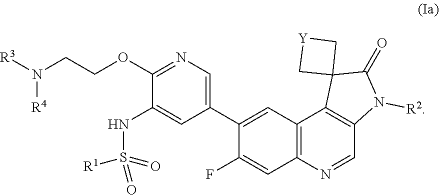

Y and R¹-R⁴ are as defined herein.

In some examples, the mesylate salt can be a mesylate salt of the compound of Formula (Ia-1):

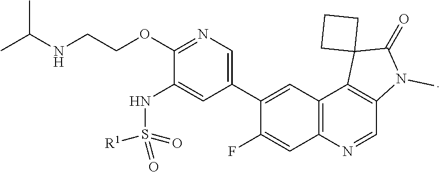

In some instances,
R¹ can be C₁₋₃ alkyl. For example, the mesylate salt has the following structure:

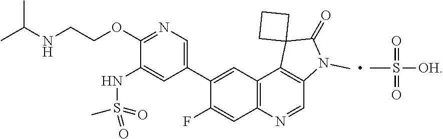

In other examples, the mesylate salt can be a mesylate salt of the compound of Formula (Ib):

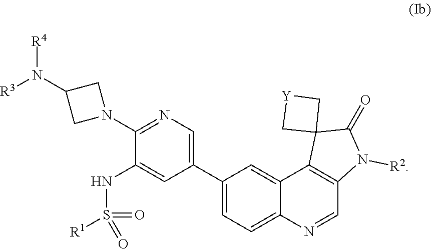

Y and R¹⁻⁴ are as defined herein.

Further, the instant disclosure provides a pharmaceutical composition, comprising a mesylate salt of any of the Formula (I) compounds disclosed herein. In addition, the present disclosure provides a method for treating cancer, comprising administering to a subject in need thereof an effective amount of the mesylate salt disclosed herein, or the pharmaceutical composition comprising such.

Also within the scope of the present disclosure are co-use of any of the Formula (I) compounds (e.g., in mesylate salt form) and the immune checkpoint inhibitor, optionally with a radiotherapy, for use in treating a target cancer as those disclosed herein, or co-use of such therapeutic agents for manufacturing a medicament for use in treating the target cancer. In addition, the present disclosure provides pharmaceutical compositions comprising any of the mesylate salt as disclosed herein for use in treating a target cancer and use of such mesylate salts for manufacturing a medicament for use in treating the target cancer.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is an image of an immunoblot from a representative experiment assessing the effect of Compound 569 on MCF7 cells with or without radiation.

FIGS. 2A-2B include graphs showing that Compound 569 improved cell viability in a clonogenic assay. FIG. 2A: a graph showing the results of a clonogenic survival assay examining the MCF7 cell viability after exposure to vehicle (DMSO) or Compound 569 with or without radiation. FIG. 2B: a graph showing the results of a clonogenic survival assay examining the A549 cell viability after exposure to vehicle (DMSO) or Compound 569 with or without radiation.

FIG. 3 is an image of an immunoblot showing the induction of phosphorylation of TBK1 by Compound 569, a selective ATM inhibitor (ATMi), a selective DNA-PK inhibitor (DNA-PKi), and a combination of the selective ATM inhibitor and the selective DNA-PK inhibitor (Ai+Di) in HCT116 cells expressing wild-type p53 or HCT116 cells that were negative for p53 expression. In FIG. 3, p.ATM, p.DNA-PK, p.TBK1, and p.STING indicate phosphorylated forms of ATM, DNA-PK, TBK1, and STING, respectively.

FIG. 4 is an immunoblot showing the inhibition of radiation-induced autophosphorylation of DNA-PK kinase and radiation-induced phosphorylation of KAP1, an ATM substrate, by compound 569 in FADU head and neck squamous cell carcinoma (HNSCC) human tumor xenografts. pDNA-PK and pKAP1 indicate phosphorylated forms of DNA-PK and KAP1, respectively.

FIG. 5 is an immunoblot showing the inhibition of radiation-induced autophosphorylation of DNA-PK kinase and radiation-induced phosphorylation of KAP1, an ATM substrate, by compound 569 in MDA-MB-231 breast carcinoma human tumor xenografts. pDNA-PK and pKAP1 indicate phosphorylated forms of DNA-PK and KAP1, respectively.

FIG. 6 is a diagram illustrating the dosing of a FADU subcutaneous human xenograft mouse model with compound 569 and/or IR, qd×3. The median relative tumor volume over time for each group in the study are shown. “569” means compound 569, “Veh.” means vehicle, and “Rad.” means radiation.

FIG. 7 is a plot representing the Kaplan-Meier quintupling-free survival for each group dosed in the FADU subcutaneous human xenograft mouse model with compound 569 and/or IR, qd×3. “569” means compound 569, “Veh.” means vehicle, and “Rad.” means radiation.

FIG. 8 is a diagram illustrating the dosing of a MDA-MB-231 subcutaneous human xenograft mouse model with compound 569 and/or IR, qd×3. The median relative tumor volume over time for each group in the study are shown. In FIG. 8, “569” means compound 569, “Veh.” means vehicle, and “Rad.” means radiation.

FIG. 9 is a plot representing the Kaplan-Meier quintupling-free survival data for each group dosed in the MDA-MB-231 subcutaneous human xenograft mouse model with compound 569 and/or IR, qd×3. In FIG. 9, “569” means compound 569, “Veh.” means vehicle, and “Rad.” means radiation.

FIG. 10 is a graph showing tumor growth for each group (n=10) dosed in the MC38 syngeneic mouse model with 1) vehicle+isotype control; 2) anti-mPD-1; 3) radiation+isotype control; 4) radiation+anti-mPD-1; 5) compound 569+radiation; 6) compound 569+anti-mPD-1; 7) compound 569+radiation+anti-mPD-1.

### DETAILED DESCRIPTION OF THE INVENTION

### Definition

It is to be understood that the terminology employed herein is for the purpose of describing particular embodiments, and is not intended to be limiting. Further, although any methods, devices and materials similar or equivalent to those described herein can be used in the practice or testing of the invention, the preferred methods, devices and materials are now described. In addition to the foregoing, as used in the specification and appended claims, unless specified to the contrary, the following terms have the meaning indicated:
“Amino” refers to the —NH₂ radical.
“Cyano” refers to the —CN radical.
“Hydroxyl” refers to the —OH radical.
“Imino” refers to the =NH substituent.
“Nitro” refers to the —NO₂ radical.
“Oxo” refers to the ═O substituent.

“Thioxo” refers to the =S substituent.

“Trifluoromethyl” refers to the —CF₃ radical.

“Alkyl” refers to a linear, saturated, acyclic, monovalent hydrocarbon radical or branched, saturated, acyclic, monovalent hydrocarbon radical, having from one to twelve carbon atoms, preferably one to eight carbon atoms or one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. An optionally substituted alkyl radical is an alkyl radical that is optionally substituted, valence permitting, by one, two, three, four, or five substituents independently selected from the group consisting of halo, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁵, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 or 2), —S(O)ₜOR¹⁶ (where t is 1 or 2), —S(O)ₚR¹⁶ (where p is 0, 1, or 2) and —S(O)ₜN(R¹⁴)₂ (where t is 1 or 2), where each R¹⁴ is independently hydrogen, alkyl, haloalkyl, cycloalkyl, cycloalkylalkyl, aryl, aralkyl, heterocyclyl, or heteroaryl; each R¹⁵ is independently hydrogen, cycloalkyl, aryl, heterocyclyl, or heteroaryl; and each R¹⁶ is independently alkyl, haloalkyl, cycloalkyl, aryl, heterocyclyl, or heteroaryl.

“Alkenyl” refers to a linear, acyclic, monovalent hydrocarbon radical or branched, acyclic, monovalent hydrocarbon radical, containing one, two, or three carbon-carbon double bonds, having from two to twelve carbon atoms, preferably two to eight carbon atoms and which is attached to the rest of the molecule by a single bond, e.g., ethenyl, prop-1-enyl, but-1-enyl, pent-1-enyl, penta-1,4-dienyl and the like. An optionally substituted alkenyl radical is an alkenyl radical that is optionally substituted, valence permitting, by one, two, three, four, or five substituents independently selected from the group consisting of: halo, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁵, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 or 2), —S(O)ₜOR¹⁶ (where t is 1 or 2), —S(O)ₚR¹⁶ (where p is 0, 1, or 2) and —S(O)ₜN(R¹⁴)₂ (where t is 1 or 2), where each R¹⁴ is independently hydrogen, alkyl, haloalkyl, cycloalkyl, aryl, heterocyclyl, or heteroaryl; each R¹⁵ is independently hydrogen, cycloalkyl, aryl, heterocyclyl, or heteroaryl; and each R¹⁶ is independently alkyl, haloalkyl, cycloalkyl, cycloalkylalkyl, aryl, heterocyclyl, or heteroaryl.

“Alkynyl” refers to a linear, acyclic, monovalent hydrocarbon radical or branched, acyclic, monovalent hydrocarbon radical, containing one or two carbon-carbon triple bonds and, optionally, one, two, or three carbon-carbon double bonds, and having from two to twelve carbon atoms, preferably two to eight carbon atoms and which is attached to the rest of the molecule by a single bond, e.g., ethynyl, prop-1-ynyl, but-1-ynyl, pent-1-ynyl, penta-1-en-4-ynyl and the like. An optionally substituted alkynyl radical is an alkynyl radical that is optionally substituted by one, two, three, four, or five substituents independently selected from the group consisting of: halo, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁵, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 or 2), —S(O)ₜOR¹⁶ (where t is 1 or 2), —S(O)ₚR¹⁶ (where p is 0, 1, or 2) and —S(O)ₜN(R¹⁴)₂ (where t is 1 or 2) where each R¹⁴ is independently hydrogen, alkyl, haloalkyl, cycloalkyl, aryl, heterocyclyl, or heteroaryl; each R¹⁵ is independently hydrogen, cycloalkyl, aryl, heterocyclyl, or heteroaryl; and each R¹⁶ is independently alkyl, haloalkyl, cycloalkyl, aryl, heterocyclyl, or heteroaryl.

“Alkylene” or “alkylene chain” refers to a linear, acyclic, saturated, divalent hydrocarbon chain or branched, acyclic, saturated, divalent hydrocarbon chain, having from one to twelve carbon atoms, e.g., methylene, ethylene, propylene, n-butylene, and the like. The alkylene chain is attached through single bonds. The points of attachment of the alkylene chain may be on the same carbon atom or on different carbon atoms within the alkylene chain. An optionally substituted alkylene chain is an alkylene chain that is optionally substituted, valence permitting, by one, two, three, four, or five substituents independently selected from the group consisting of: halo, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁵, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 or 2), —S(O)ₜOR¹⁶ (where t is 1 or 2), —S(O)ₚR¹⁶ (where p is 0, 1, or 2) and —S(O)ₜN(R¹⁴)₂ (where t is 1 or 2) where each R¹⁴ is independently hydrogen, alkyl, haloalkyl, cycloalkyl, aryl, heterocyclyl, or heteroaryl; each R¹⁵ is independently hydrogen, cycloalkyl, aryl, heterocyclyl, or heteroaryl; and each R¹⁶ is independently alkyl, haloalkyl, cycloalkyl, aryl, heterocyclyl, or heteroaryl. In some embodiments, alkylene is ethylene.

“Alkenylene” or “alkenylene chain” refers to a linear, acyclic, divalent hydrocarbon chain or branched, acyclic, divalent hydrocarbon chain, containing one, two, or three carbon-carbon double bonds and having from two to twelve carbon atoms, e.g., ethenylene, propenylene, n-butenylene and the like. The alkenylene chain is attached through single bonds. The points of attachment of the alkenylene chain may be on the same carbon atom or on different carbon atoms within the alkenylene chain. An optionally substituted alkenylene chain is an alkenylene chain that is optionally substituted, valence permitting, by one, two, three, four, or five substituents independently selected from the group consisting of: halo, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁵, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 or 2), —S(O)ₜOR¹⁶ (where t is 1 or 2), —S(O)ₚR¹⁶ (where p is 0, 1, or 2) and —S(O)ₜN(R¹⁴)₂ (where t is 1 or 2) where each R¹⁴ is independently hydrogen, alkyl, haloalkyl, cycloalkyl, aryl, heterocyclyl, or heteroaryl; each R¹⁵ is independently hydrogen, cycloalkyl, aryl, heterocyclyl, or heteroaryl; and each R¹⁶ is independently alkyl, haloalkyl, cycloalkyl, aryl, heterocyclyl, or heteroaryl.

“Alkynylene” or “alkynylene chain” refers to a linear, acyclic, divalent, hydrocarbon chain or branched, acyclic, divalent hydrocarbon chain, containing one or two carbon-carbon triple bonds and, optionally, one, two, or three carbon-carbon double bonds, and having from two to twelve carbon atoms, e.g., propynylene, n-butynylene and the like. The alkynylene chain is attached through single bonds. The points of attachment of the alkynylene may be on the same carbon atom or on different carbon atoms within the alkynylene chain. An optionally substituted alkynylene chain is an alkynelene chain that is optionally substituted by one, two, three, four, or five substituents independently selected from the group consisting of: halo, cyano, nitro, aryl, cycloalkyl, heterocyclyl, heteroaryl, oxo, trimethylsilanyl, —OR¹⁴, —OC(O)—R¹⁴, —N(R¹⁴)₂, —C(O)R¹⁵, —C(O)OR¹⁴, —C(O)N(R¹⁴)₂, —N(R¹⁴)C(O)OR¹⁶, —N(R¹⁴)C(O)R¹⁶, —N(R¹⁴)S(O)ₜR¹⁶ (where t is 1 or 2), —S(O)ₜOR¹⁶ (where t is 1 or 2), —S(O)ₚR¹⁶ (where p is 0, 1, or 2) and —S(O)ₜN(R¹⁴)₂ (where t is 1 to 2) where each R¹⁴ is independently hydrogen, alkyl, haloalkyl, cycloalkyl, aryl, heterocyclyl, or heteroaryl; each R¹⁵ is independently hydrogen, cycloalkyl, aryl, heterocyclyl, or heteroaryl; and each R¹⁶ is independently alkyl, haloalkyl, cycloalkyl, aryl, heterocyclyl, or heteroaryl.

“Alkoxy” refers to a radical of the formula —ORₐ where Rₐ is an alkyl radical as defined above containing one to twelve carbon atoms. The alkyl part of the optionally substituted alkoxy radical is optionally substituted as defined above for an alkyl radical.

“Alkoxyalkyl” refers to a radical of the formula —Rₐ—O—R_{b} where Rₐ is alkylene and R_{b} is alkyl as defined above. Alkyl and alkylene parts of the optionally substituted alkoxyalkyl radical are optionally substituted as defined above for an alkyl radical and alkylene chain, respectively.

“Aralkyl” refers to a radical of the formula —Rₐ—R_{b}, where Rₐ is alkylene and R_{b} is aryl as described herein. Alkylene and aryl portions of optionally substituted aralkyl are optionally substituted as described herein for alkylene and aryl, respectively.

“Aryl” refers to an aromatic monocyclic or multicyclic hydrocarbon ring system radical containing from 6 to 18 carbon atoms, where the multicyclic aryl ring system is a bicyclic, tricyclic, or tetracyclic ring system. Aryl radicals include, but are not limited to, groups such as fluorenyl, phenyl and naphthyl. An optionally substituted aryl is an aryl radical that is optionally substituted by one, two, three, four, or five substituents independently selected from the group consisting of alkyl, akenyl, halo, haloalkyl, haloalkenyl, cyano, nitro, aryl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 or 2), —R¹⁵—S(O)ₜOR¹⁶ (where t is 1 or 2), —R¹⁵—S(O)ₚR¹⁶ (where p is 0, 1, or 2), and —R¹⁵—S(O)ₜN(R¹⁴)₂ (where t is 1 or 2), where each R¹⁴ is independently hydrogen, alkyl, haloalkyl, cycloalkyl, aryl, heterocyclyl, or heteroaryl; each R¹⁵ is independently a direct bond or a linear or branched alkylene or alkenylene chain; and each R¹⁶ is independently alkyl, haloalkyl, cycloalkyl, cycloalkylalkyl, aryl, heterocyclyl, or heteroaryl.

“Arylalkoxy” refers to a group of formula —O—R, where R is aralkyl. An optionally substituted arylalkoxy is an arylalkoxy that is optionally substituted as described herein for aralkyl.

In some embodiments, arylalkoxy is benzyloxy. “Cycloalkyl” refers to a stable non-aromatic monocyclic or polycyclic hydrocarbon radical having from three to fifteen carbon atoms, preferably having from three to ten carbon atoms, and which is saturated or unsaturated, and which attaches to the rest of the molecule by a single bond. A polycyclic hydrocarbon radical is bicyclic, tricyclic, or tetracyclic ring system. An unsaturated cycloalkyl contains one, two, or three carbon-carbon double bonds and/or one carbon-carbon triple bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, and the like. An optionally substituted cycloalkyl is a cycloalkyl radical that is optionally substituted by one, two, three, four, or five substituents independently selected from the group consisting of alkyl, alkenyl, halo, haloalkyl, haloalkenyl, cyano, nitro, oxo, aryl, aralkyl, cycloalkyl, heterocyclyl, heteroaryl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 or 2), —R¹⁵—S(O)ₜOR¹⁶ (where t is 1 or 2), —R¹⁵—S(O)ₚR¹⁶ (where p is 0, 1, or 2) and —R¹⁵—S(O)ₜN(R¹⁴)₂ (where t is 1 or 2) where each R¹⁴ is independently hydrogen, alkyl, haloalkyl, cycloalkyl, aryl, heterocyclyl, or heteroaryl; each R¹⁵ is independently a direct bond or a linear or branched alkylene or alkenylene chain; and each R¹⁶ is independently alkyl, haloalkyl, cycloalkyl, cycloalkylalkyl, aryl, aralkyl, heterocyclyl, or heteroaryl.

“Fused” refers to any ring system described herein which is fused to an existing ring structure in the compounds of the invention. When the fused ring system is a heterocyclyl or a heteroaryl, any carbon atom on the existing ring structure which becomes part of the fused ring system may be replaced with a nitrogen atom.

“Halo” refers to the halogen substituents: bromo, chloro, fluoro, and iodo.

“Haloalkyl” refers to an alkyl radical, as defined above, that is further substituted by one or more halogen substituents. The number of halo substituents included in haloalkyl is from one and up to the total number of the hydrogen atoms available for replacement with the halo substituents (e.g., perfluoroalkyl). Non-limiting examples of haloalkyl include trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl and the like. For an optionally substituted haloalkyl, the hydrogen atoms bonded to the carbon atoms of the alkyl part of the haloalkyl radical may be optionally replaced with substituents as defined above for an optionally substituted alkyl.

“Haloalkenyl” refers to an alkenyl radical, as defined above, that is further substituted by one or more halo substituents. The number of halo substituents included in haloalkenyl is from one and up to the total number of the hydrogen atoms available for replacement with the halo substituents (e.g., perfluoroalkenyl). Non-limiting examples of haloalkenyl include 2,2-difluoroethenyl, 3-chloroprop-1-enyl, and the like. For an optionally substituted haloalkenyl, the hydrogen atoms bonded to the carbon atoms of the alkenyl part of the haloalkenyl radical may be optionally replaced with substitutents as defined above for an optionally substituted alkenyl group.

“Haloalkynyl” refers to an alkynyl radical, as defined above, that is further substituted by one or more halo substituents. The number of halo substituents included in haloalkynyl is from one and up to the total number of the hydrogen atoms available for replacement with the halo substituents (e.g., perfluoroalkynyl). Non-limiting examples of haloalkynyl include 3-chloroprop-1-ynyl and the like. The alkynyl part of the haloalkynyl radical may be additionally optionally substituted as defined above for an alkynyl group.

“Heteroarylalkyl” refers to a radical of the formula —Rₐ—R_{b}, where Rₐ is alkylene and R_{b} is heteroaryl as described herein. Alkylene and heteroaryl portions of optionally substituted heteroarylalkyl are optionally substituted as described herein for alkylene and heteroaryl, respectively.

“Heterocyclyl” refers to a stable 3- to 18-membered non-aromatic ring system radical having the carbon count of two to twelve and containing a total of one to six heteroatoms independently selected from the group consisting of nitrogen, oxygen, phosphorus, and sulfur. A heterocyclyl radical is a monocyclic, bicyclic, tricyclic, or tetracyclic ring system. A bicyclic, tricyclic, or tetracyclic heterocyclyl is a fused, spiro, and/or bridged ring system. The heterocyclyl radical may be saturated or unsaturated. An unsaturated heterocyclyl contains one, two, or three carbon-carbon double bonds and/or one carbon-carbon triple bond. An optionally substituted heterocyclyl is a heterocyclyl radical that is optionally substituted by one, two, three, four, or five substituents independently selected from the group consisting of alkyl, alkenyl, halo, haloalkyl, haloalkenyl, cyano, oxo, thioxo, nitro, aryl, aralkyl, cycloalkyl, heterocyclyl, heteroaryl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 or 2), —R¹⁵—S(O)ₜOR¹⁶ (where t is 1 or 2), —R¹⁵—S(O)ₚR¹⁶ (where p is 0, 1, or 2), and —R¹⁵—S(O)ₜN(R¹⁴)₂ (where t is 1 or 2), where each R¹⁴ is independently hydrogen, alkyl, alkenyl, haloalkyl, cycloalkyl, aryl, heterocyclyl, or heteroaryl; each R¹⁵ is independently a direct bond or a linear or branched alkylene or alkenylene chain; and each R¹⁶ is independently alkyl, alkenyl, haloalkyl, cycloalkyl, aryl, heterocyclyl, or heteroaryl. The nitrogen, carbon, or sulfur atoms in the heterocyclyl radical may be optionally oxidized (when the substituent is oxo and is present on the heteroatom); the nitrogen atom may be optionally quaternized (when the substituent is alkyl, alkenyl, aryl, aralkyl, cycloalkyl, heterocyclyl, heteroaryl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 or 2), —R¹⁵—S(O)ₜOR¹⁶ (where t is 1 or 2), —R¹⁵—S(O)ₚR¹⁶ (where p is 0, 1, or 2), and —R¹⁵—S(O)ₜN(R¹⁴)₂ (where t is 1 or 2), where R¹⁵ is a linear or branched alkylene or alkenylene chain, and R¹⁴ and R¹⁶ are as defined above). Examples of optionally substituted heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“Heterocyclylene” refers to a heterocyclyl in which one hydrogen atom is replaced with a valency. An optionally substituted heterocyclylene is optionally substituted as described herein for heterocyclyl.

“Heteroaryl” refers to a 5- to 18-membered ring system radical containing at least one aromatic ring, having the carbon count of one to seventeen carbon atoms, and containing a total of one to ten heteroatoms independently selected from the group consisting of nitrogen, oxygen, and sulfur. The heteroaryl radical is a monocyclic, bicyclic, tricyclic, or tetracyclic ring system. The bicyclic, tricyclic, or tetracyclic heteroaryl radical is a fused and/or bridged ring system. An optionally substituted heteroaryl is a heteroaryl radical that is optionally substituted by one, two, three, four, or five substituents independently selected from the group consisting of alkyl, alkenyl, alkoxy, halo, haloalkyl, haloalkenyl, cyano, oxo, thioxo, nitro, oxo, aryl, aralkyl, cycloalkyl, heterocyclyl, heteroaryl, or heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 or 2), —R¹⁵—S(O)ₜOR¹⁶ (where t is 1 or 2), —R¹⁵—S(O)ₜR¹⁶ (where p is 0, 1, or 2), and —R¹⁵—S(O)ₜN(R¹⁴)₂ (where t is 1 or 2), where each R¹⁴ is independently hydrogen, alkyl, alkenyl, haloalkyl, cycloalkyl, aryl, heterocyclyl, or heteroaryl; each R¹⁵ is independently a direct bond or a linear or branched alkylene or alkenylene chain; and each R¹⁶ is alkyl, alkenyl, haloalkyl, cycloalkyl, aryl, heterocyclyl, or heteroaryl. The nitrogen, carbon, or sulfur atoms in the heterocyclyl radical may be optionally oxidized (when the substituent is oxo and is present on the heteroatom), provided that at least one ring in heteroaryl remains aromatic; the nitrogen atom may be optionally quaternized (when the substituent is alkyl, alkenyl, aryl, aralkyl, cycloalkyl, heterocyclyl, heteroaryl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)S(O)ₜR¹⁶ (where t is 1 or 2), —R¹⁵—S(O)ₜOR¹⁶ (where t is 1 or 2), —R¹⁵—S(O)ₚR¹⁶ (where p is 0, 1, or 2), and —R¹⁵—S(O)ₜN(R¹⁴)₂ (where t is 1 or 2), where R¹⁵ is a linear or branched alkylene or alkenylene chain, and R¹⁴ and R¹⁶ are as defined above), provided that at least one ring in heteroaryl remains aromatic. Examples of optionally substituted heteroaryl radicals include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyl, naphthyridinyl, oxadiazolyl, 2-oxoazepinyl, oxazolyl, oxiranyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrrolyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl and thiophenyl (i.e., thienyl).

### I. Cancer Treatment

The ATM (ataxia-telangiectasia, mutated) and DNA-PK kinases, in particular, are important modulators of cellular responses to DNA breakage and inhibition of either of these molecules markedly increases the sensitivity of cells to ionizing radiation. Humans and mice having loss-of-function mutations in the ATM or PRKDC genes, which encode Ataxia Telangiectasia Mutated (ATM) kinase and DNA-dependent Protein Kinase (DNA-PK), respectively, are hypersensitive to ionizing radiation. Inhibition of ATM and DNA-PK kinases together can be effective in sensitizing tumor cells to radiation or DNA damaging agents (e.g., anti-tumor agents). The efficacy of dual inhibition of ATM and DNA-PK kinases may be superior to inhibition of either kinase by itself.

Immunotherapy is a type of cancer treatment by modulating a patient's own immune system, leading to enhanced anti-cancer cell immune responses. Immunotherapy showed success in prolonging progression-free survival and overall survival rates in cancer patients. On the other hand, immunotherapy may cause severe adverse events due to an overactive immune system.

The present disclosure provides combined cancer therapy comprising (a) one or more compounds of Formula (I) or a pharmaceutically acceptable salt thereof (e.g., Compound 568, 569, 570, or 574, or a pharmaceutically acceptable salt thereof such as a mesylate salt or an HCl salt), (b) one or more immune checkpoint inhibitors such as PD1 antagonists, and optionally (c) a radiotherapy. As reported herein, the Formula (I) compounds are selective, dual inhibitors of ATM (ataxia-telangiectasia, mutated) and DNA-PK kinases but have low or no inhibitory activity against kinases such as that inhibits mTOR, and/or PI3Kα/δ, and/or have low or no inhibitory activity against hERG. Thus, the compounds of Formula (I) disclosed herein can be effective inhibitors of the actions of ATM and DNA-PK with or without radiation. Further, the combination of Formula (I) compounds and immunotherapy (e.g., immune checkpoint inhibitors) would be expected to achieve superior anti-tumor effects, for example, by stimulating pathways involved in activating anti-tumor immune signaling pathways. Without being bound by theory, the combined therapy may, in some instances, achieve the intended therapeutic effects by activating tumor-cell specific T cells (via, e.g., changing the cytokine profile within the tumor microenvironment, promoting immunogenic cell death, stimulating neoantigen presentation, or a combination thereof). Additionally, the combination of Formula (I) compounds and immunotherapy with radiation could enhance that response by similar or other multiple mechanisms.

### A. Therapeutic Agents

The combined therapy disclosed herein comprise one or more of the Formula (I) compounds or a pharmaceutically acceptable salt thereof as disclosed herein (e.g., Compound 568, 569, 570, or 574, or a pharmaceutically acceptable salt thereof), one or more immunotherapeutics (e.g., immune checkpoint inhibitors such as PD1 antagonists, and optionally radiotherapy). The compound disclosed herein encompasses any stereoisomer, enantiomer, tautomer, or a mixture thereof.

### (i) Compound of Formula (I)

In some aspects, provided herein are dual ATM/DNA-PK inhibitory compounds having the structure set forth as Formula (I):

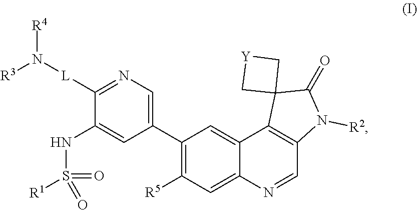

or a pharmaceutically acceptable salt thereof. In Formula (I), R⁵ can be H or halogen (e.g., fluoro); Y and be CHR⁶ or NR⁷; L can be —OR⁸—, or —N(R⁸)₂—. Each R⁸ may independently be H or C₁₋₃ alkyl. In some instances, both R⁸ substituents, together with N, form a heterocyclyl ring; and R¹, R², R³, R⁴, R⁶, and R⁷ are each independently H, or C₁₋₃ alkyl (substituted or unsubstituted). In some examples, C₁₋₃ alkyl can be methyl, ethyl, propyl, or isopropyl.

In some embodiments, L is be —OR⁸—, in which R⁸ is H. In some embodiments, L is be —OR⁸—, in which R⁸ or C₁₋₃ alkyl.

In some embodiments, Y in Formula (I) may be CHR⁵. In some examples, R⁵ can be H.

In other examples, R⁵ can be C₁₋₃ alkyl (e.g., unsubstituted C₁₋₃ alkyl such as methyl, ethyl, propyl or isopropyl). Alternatively or in addition, each of R², R³, and R⁴ may be H. In other examples, each of R², R³, and R⁴ may be C₁₋₃ alkyl (e.g., unsubstituted C₁₋₃ alkyl such as methyl, ethyl, propyl or isopropyl). Alternatively or in addition, R¹ can be methyl. In other examples, R¹ can be ethyl. In yet other examples, R¹ can be isopropyl.

In some embodiments, Y in Formula (I) may be NR⁶. In some examples, R⁶ can be H. In other examples, R⁶ can be C₁₋₃ alkyl (e.g., unsubstituted C₁₋₃ alkyl such as methyl, ethyl, or propyl). Alternatively or in addition, each of R², R³, and R⁴ may be H. In other examples, each of R², R³, and R⁴ may be C₁₋₃ alkyl (e.g., unsubstituted C₁₋₃ alkyl such as methyl, ethyl, or propyl). Alternatively or in addition, R¹ can be methyl. In other examples, R¹ can be ethyl. In yet other examples, R¹ can be isopropyl.

In some embodiments, the dual ATM/DNA-PK inhibitory compounds disclosed herein may have the structure of Formula (Ia):

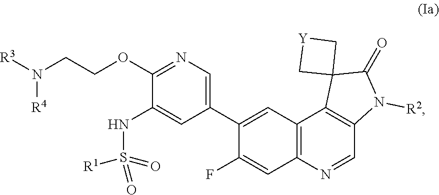

in which Y and R¹-R⁴ are as defined herein. In some examples, the dual ATM/DNA-PK inhibitory compounds may be a compound of Formula (Ia-1):

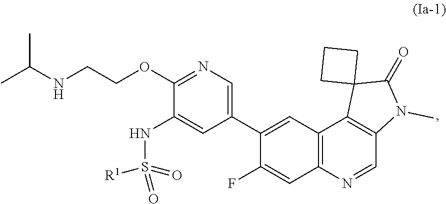

in which R¹ is as defined herein.

In some embodiments, the dual ATM/DNA-PK inhibitory compounds disclosed herein may have the structure of Formula (Ib):

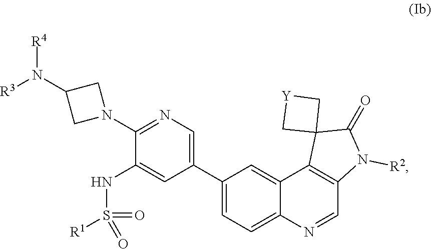

wherein Y and R¹⁻⁴ are as defined herein.

The compounds of Formula (I) disclosed herein can be dual inhibitors of ATM and DNA-PK kinases. For example, a Formula (I) compound disclosed herein may have an IC₅₀ value against ATM ≤5 nM and/or an IC₅₀ value against DNA-PK≤1.1 nM (e.g., ≤1.0 nM) (e.g., Compound 568, 569, 570, or 574). In some instances, the compounds of Formula (I) disclosed herein are selective inhibitors against ATM and DNA-PK. Such selective inhibitors have significantly reduced (or no) inhibitory activity against other target enzymes such as mTOR, PI3K-α/δ, and/or hERG. For example, a compound of Formula (I) disclosed herein (e.g., Compound 568, 569, 570, or 574) may have an mTOR IC₅₀ of at least 10 times (e.g., at least 20 times) greater than the ATM IC₅₀ or DNA-PK IC₅₀. In other examples, a Formula (I) compound (e.g., Compound 568, 569, 570, or 574) may have an mTOR IC₅₀ of 10 nM or greater (e.g., >100 nM). Additionally or alternatively, a compound of Formula (I) (e.g., Compound 568, 569, 570, or 574) may have an hERG IC₅₀ of at least 100 times (e.g., at least 500 times, at least 1000 times, or at least 3000 times) greater than the ATM IC₅₀ or DNA-PK IC₅₀, when measured at the same compound concentration. For example, a compound of Formula (I) (e.g., Compound 568, 569, 570, or 574) may have an hERG IC₅₀ of 3 μM or greater (e.g., 10 μM or greater).

Any of the Formula (I) compounds disclosed herein may be used in the combined cancer therapy disclosed herein. Exemplary compounds of Formula (I) for use in include, but are not limited to:
trans-N-(5-(3-Ethyl-7′-fluoro-3′-methyl-2′-oxo-2′,3′-dihydrospiro[cyclobutane-1,1′-pyrrolo[2,3-c]quinolin]-8′-yl)-2-(2-(isopropylamino)ethoxy)pyridin-3-yl)methanesulfonamide
N-(5-(7′-Fluoro-3′-methyl-2′-oxo-2′,3′-dihydrospiro[cyclobutane-1,1′-pyrrolo[2,3-c]quinolin]-8′-yl)-2-(2-(isopropylamino)ethoxy)pyridin-3-yl)propane-2-sulfonamide
N-(5-(7′-Fluoro-3′-methyl-2′-oxo-2′,3′-dihydrospiro[cyclobutane-1,1′-pyrrolo[2,3-c]quinolin]-8′-yl)-2-(2-(isopropylamino)ethoxy)pyridin-3-yl)methanesulfonamide hydrochloride
N-(5-(7′-Fluoro-3′-methyl-2′-oxo-2′,3′-dihydrospiro[cyclobutane-1,1′-pyrrolo[2,3-c]quinolin]-8′-yl)-2-(2-(isopropylamino)ethoxy)pyridin-3-yl)ethanesulfonamide hydrochloride
N-(5-(7′-Fluoro-1-isopropyl-3′-methyl-2′-oxo-2′,3′-dihydrospiro[azetidine-3,1-pyrrolo[2,3-c]quinolin]-8′-yl)-2-(2-(isopropylamino)ethoxy)pyridin-3-yl)methanesulfonamide
cis-N-(5-(3-Ethyl-7′-fluoro-3′-methyl-2′-oxo-2′,3′-dihydrospiro[cyclobutane-1,1-pyrrolo[2,3-c]quinolin]-8′-yl)-2-(2-(isopropylamino)ethoxy)pyridin-3-yl)methanesulfonamide
N-(2-(3-(Dimethylamino)azetidin-1-yl)-5-(3′-methyl-2′-oxo-2′,3′-dihydrospiro[cyclobutane-1,1′-pyrrolo[2,3-c]quinolin]-8′-yl)pyridin-3-yl)methanesulfonamide hydrochloride

In specific examples, the compound of Formula (I) for use in the combined therapy is Compound 568. In other examples, the compound of Formula (I) is Compound 569. In yet other examples, the compound of Formula (I) is Compound 570. In other examples, the compound of Formula (I) is Compound 574.

In some instances, a pharmaceutically acceptable salt of a Formula (I) compound can be used in the combined cancer therapy disclosed herein. “Pharmaceutically acceptable salt,” as used herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Pharmaceutically acceptable salts include acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid and the like.”

Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some examples, a mesylate salt of a Formula (I) compound (e.g., Compound 568, 569, 570, or 574) can be used in the combined therapy disclosed herein. One specific example is provided below:

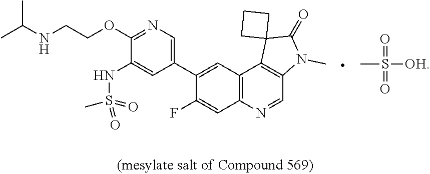

The Formula (I) compounds disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and ( ), (R)- and (S), or (D)- and (L)-isomers may be prepared using chiral synthons or chiral reagents, or resolved using conventional techniques, for example, chromatography and fractional crystallisation. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer refers to a proton shift from one atom of a molecule to another atom of the same molecule. Tautomers of any Formula (I) compounds disclosed herein are within the scope of the present disclosure.

Also within the scope of the present disclosure are intermediate compounds of formula (I) and all polymorphs of the aforementioned species and crystal habits thereof.

### Preparation of Formula (I) Compounds

The Formula (I) compounds disclosed herein can be prepared using methods and techniques known in the art or follow disclosures provided herein (e.g., see Examples below). Suitable processes for synthesizing these compounds are provided in the Examples. Generally, compounds of Formula (I) can be prepared according to the Schemes described below. The sources of the starting materials for these reactions are also described.

Protecting groups may be added or removed in the preparation of the compounds of the invention in accordance with standard techniques, which are known to one skilled in the art and as described herein. The use of protecting groups is described in detail in Greene, T. W. and P. G. M. Wuts, Greene's Protective Groups in Organic Synthesis (2006), 4^{th} Ed., Wiley. The protecting group may also be a polymer resin such as a Wang resin or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of the invention which are pharmacologically active.

All of the compounds described below as being prepared which may exist in free base or acid form may be converted to their pharmaceutically acceptable salts by treatment with the appropriate inorganic or organic base or acid. Salts of the compounds prepared below may be converted to their free base or acid form by standard techniques. It is understood that all polymorphs, amorphous forms, anhydrates, hydrates, solvates and salts of the compounds of the invention are intended to be within the scope of the invention. Furthermore, all compounds of the invention which contain an acid or an ester group can be converted to the corresponding ester or acid, respectively, by methods known to one skilled in the art or by methods described herein.

A general representation of preparation of many of these compounds is shown below in Scheme 1. Compounds are prepared through the coupling of various components of the molecule: Suzuki coupling of halo substituted compound 3 (or 2′) with a boronic acid or borate compound 2 (or 3′). Further reactions may or may not be needed to furnish the synthesis of the compounds of this invention. Preparations of specific compounds of this invention are shown in the following Schemes.

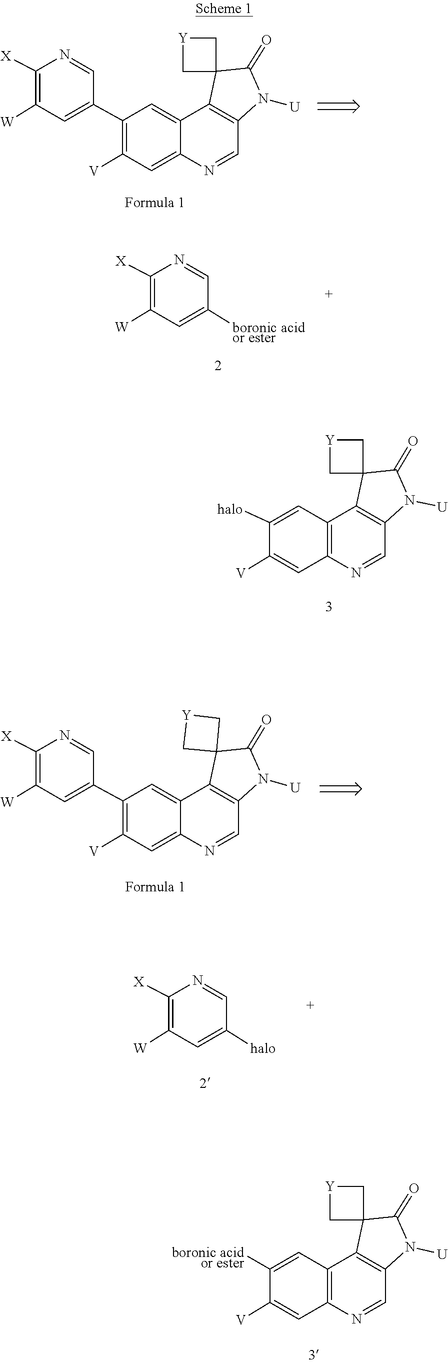

In aryl-aryl coupling reactions, halogen may be iodo, bromo, or chloro, preferable bromo or iodo. In this method, halogen substitutions may be transformed to aryl substitutions using Suzuki coupling reaction conditions. The conditions of this method are disclosed in many publications which have been reviewed by A. Suzuki in an article entitled “The Suzuki reaction with arylboron compounds in arene chemistry” in Modern Arene Chemistry 2002, 53-106. In carrying out this reaction any of the suitable conditions conventional in a Suzuki reaction can be utilized. Generally, Suzuki coupling reactions are carried out in the presence of a transition metal catalyst such as a palladium catalyst utilizing any conventional organic solvent for this reaction and a weak inorganic or organic base. Among the preferred organic solvents are the polar aprotic solvents. Any conventional polar aprotic solvents can be utilized in preparing compounds of the invention. Suitable solvents are customary, especially higher-boiling solvents, e.g. dimethoxyethane. The weak inorganic base can be a carbonate or bicarbonate, such as potassium carbonate or cesium carbonate. The organic base can be an amine such as triethylamine.

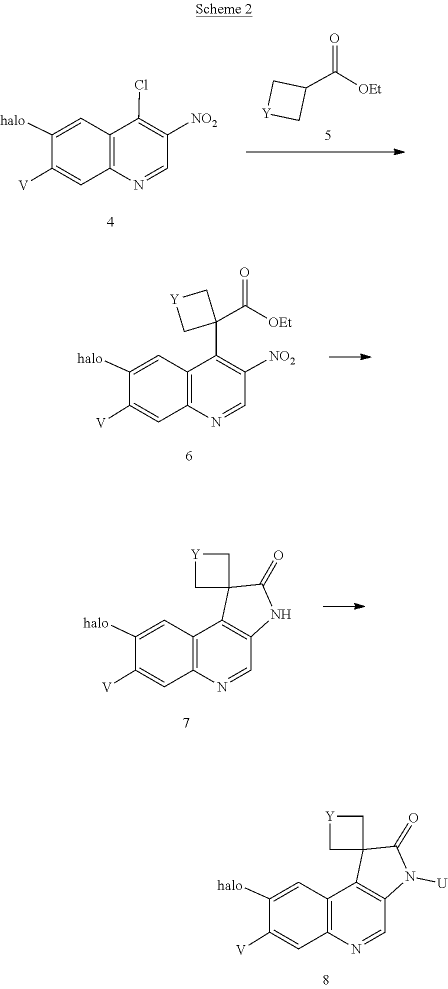

Specifically, the other spiro oxindole intermediate 7 is synthesized as shown in Scheme 2. The cyclyl or heterocyclyl substituted ester 5 is treated with a strong base such as, but not limited to, lithium diisopropylamide at low temperature in anhydrous solvent such as, but not limited to, tetrahydrofuran to react with starting material 4, which is either commercially available or prepared by those skilled in the art following the literature described methods to provide intermediate 6. Intermediate 6 is reduced by a reducing reagent such as, but not limited to, iron to give the corresponding amino intermediate which cyclizes to provide the oxindole compound 7 in situ. Thus, the compound 7 is then N-alkylated with an alkylating reagent in the presence of a base such as, but not limited to, potassium carbonate or sodium hydride in a polar solvent such as, but not limited to, N,N-dimethylformamide or tetrahydrofuran thereby to generate the spiro oxindole intermediate 8.

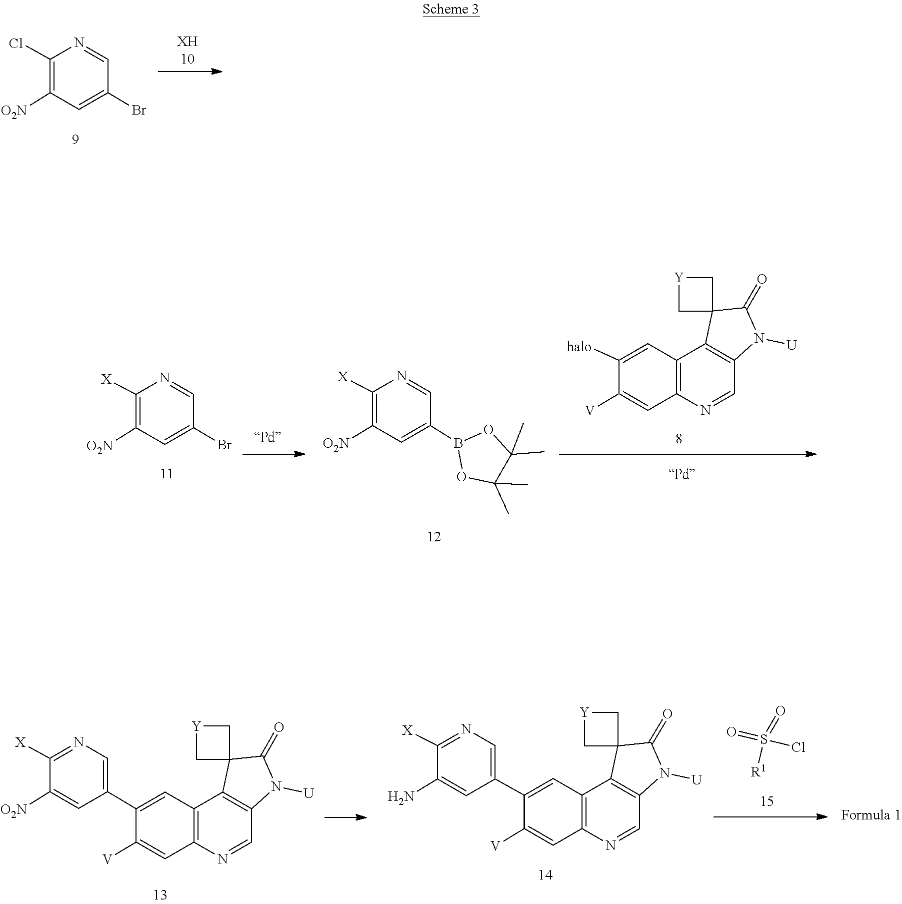

Specifically, the compounds of Formula (I) in this invention can be synthesized as shown in Scheme 3. Commercially available 5-bromo-2-chloro-3-nitro-pyridine (9) reacts with a nucleophile XH (10) in the presence of a strong base such as, but not limited to, sodium hydride to provide intermediate 11. Under palladium catalyzed conditions, borate 12 can be prepared, which then reacts with the spiro intermediate 8 to provide the cross coupled product 13. The nitro group in compound 13 is reduced to amino group using a reducing reagent such as, but not limited to, iron to provide intermediate 14. Reaction of 14 with different sulphonyl chlorides (15) furnishes the synthesis of compounds of Formula (I).

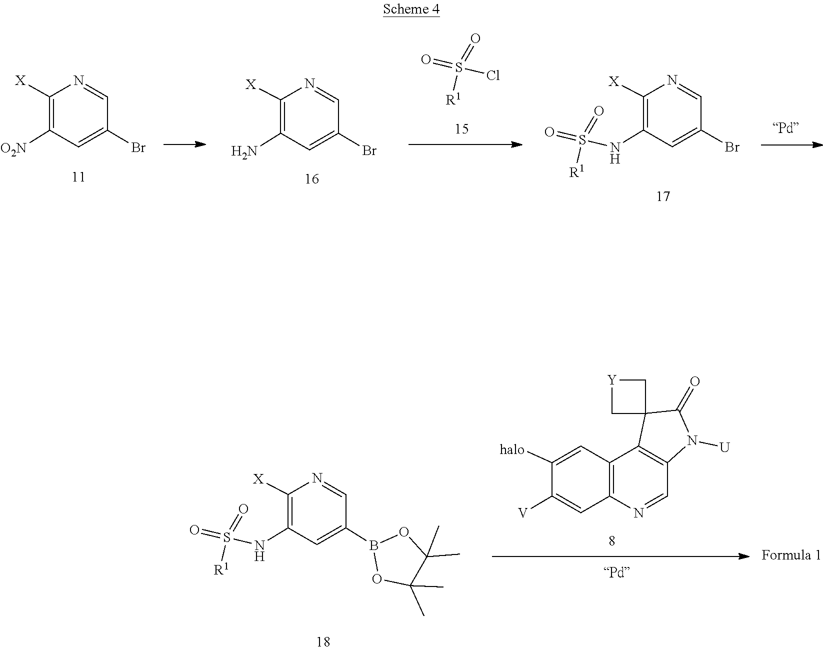

Specifically, the compounds of Formula (I) in this invention can also be synthesized as shown in Scheme 4. The nitro group in compound 11 is reduced to amino group using a reducing reagent such as, but not limited to, iron to provide intermediate 16. Reaction of 16 with different sulphonyl chlorides (15) provides the sulphonamide intermediate 17, which is converted to its corresponding borate 18 under palladium catalysis. Borate 18 can couple with the halo compound 8 under Suzuki reaction conditions to provide the compounds of Formula (I).

In Scheme 3 and Scheme 4, the cross coupled compounds are also synthesizable using Suzuki coupling chemistry with components having reversed the halogen and boronate/boronic acid substitution patterns, for example, as shown in Scheme 5.

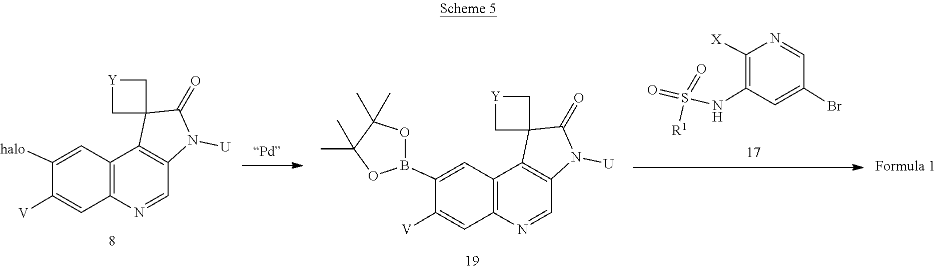

Specifically, the compounds of Formula (I) in this invention can also be synthesized as shown in Scheme 5. The halo compound 8 can be converted to its corresponding borate 19 under palladium catalysis. Borate 19 can couple with the halo compound 17 under Suzuki reaction conditions to provide the compounds of Formula (I).

### (ii) Immune Checkpoint Inhibitors

The combined cancer therapy disclosed herein further comprises one or more immunotherapeutic agents. In some embodiments, the immunotherapeutic agent is an immune checkpoint inhibitor. Immune checkpoints are regulators of the immune system that downregulate the immune response to minimize autoimmunity or an overactive immune response. Exemplary inhibitory immune checkpoints include A2AR receptor, B7-H3, B7-H4, BTLA, CTLA-4, indoleamine 2,3-dioxygenase (IDO), Killer-cell Immunoglobulin-like Receptor (KIR), Lymphocyte Activation Gene-3 (LAG3), Nicotinamide adenine dinucleotide phosphate NADPH oxidase isoform 2 (NOX2), Programed Death-1 receptor (PD-1), T-cell Immunoglobulin domain and Mucin domain 3 (Tim-3), V-domain Ig suppressor of T cell activation (VISTA), Sialic acid-binding immunoglobulin-type lectin 7 (SIGLEC7), and Sialic acid-binding immunoglobulin-type lectin 9 (SIGLEC9). Agents that block the inhibitory checkpoint molecules (e.g., those disclosed herein) are known as immune checkpoint inhibitors. Such agents can suppressive signals that block or decrease immune cell functions (e.g., T cell activities), thereby enhancing immune responses against diseased cells such as cancer cells.

The immune checkpoint inhibitors for use in the combined cancer therapy disclosed herein may be an agent that blocks any of the inhibitory checkpoint molecules disclosed herein. The checkpoint inhibitors may be an antibody, a soluble ligand of an inhibitory checkpoint receptor, or a small molecule inhibitor. Exemplary checkpoint inhibitors are provided in Table 1 below.

TABLE 1
Exemplary Immune Checkpoint Inhibitors

| Name | Molecule | Target |
|---|---|---|
| ipilimumab | Monoclonal antibody | CTLA-4 |
| tremelimumab | Monoclonal antibody | CTLA-4 |
| AMP-224 | PD-L2 Fusion protein | PD-1 |
| AMP-514 | Monoclonal antibody | PD-1 |
| AUNP-12 | Small branched peptide | PD-1 |
| camrelizumab | Monoclonal antibody | PD-1 |
| cemiplimab | Monoclonal antibody | PD-1 |
| dostarlimab | Monoclonal antibody | PD-1 |
| INCMGA00012 | Monoclonal antibody | PD-1 |
| nivolumab | Monoclonal antibody | PD-1 |
| pembrolizumab | Monoclonal antibody | PD-1 |
| retifanlimab | Monoclonal antibody | PD-1 |
| spartalizumab | Monoclonal antibody | PD-1 |
| sintilimab | Monoclonal antibody | PD-1 |
| tislelizumab | Monoclonal antibody | PD-1 |
| toripalimab | Monoclonal antibody | PD-1 |
| atezolizumab | Monoclonal antibody | PD-L1 |
| avelumab | Monoclonal antibody | PD-L1 |
| BMS-986189 | Macrocyclic peptide | PD-L1 |
| CA-170 | Small molecule | PD-L1 |
| cosibelimab | Monoclonal antibody | PD-L1 |
| durvalumab | Monoclonal antibody | PD-L1 |
| Envafolimab | Single-domain antibody | PD-L1 |
| INCB86550 | Small molecule | PD-L1 |
| REGN3504 | Monoclonal antibody | PD-L1 |

In some embodiments, the immune checkpoint inhibitor disclosed herein targets CTLA-4, for example, an antibody specific to CTLA-4 (anti-CTLA-4 antibody).

In some embodiments, the immune checkpoint inhibitor is a PD1 antagonist, which can be any agent that inhibits the signaling pathway mediated by the PD-1/PD-L1 interaction. In some examples, the PD1 antagonist can be an anti-PD1 antibody. In other examples, the PD1 antagonist can be an anti-PD-L1 antibody.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-PD1 or anti-PD-L1 antibody, encompasses not only intact (e.g., full-length) polyclonal or monoclonal antibodies, but also antigen-binding fragments thereof (such as Fab, Fab′, F(ab′)2, Fv), single-chain antibody (scFv), fusion proteins comprising an antibody portion, humanized antibodies, chimeric antibodies, diabodies, single domain antibody (e.g., nanobody), single domain antibodies (e.g., a V_{H} only antibody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

In some embodiments, the immune checkpoint inhibitor for use in the combined therapy can be an anti-PD-1 antibody listed in Table 1 above (e.g., cemiplimab or pembrolizumab), or a functional variant thereof. In other embodiments, the immune checkpoint inhibitor fur use in the combined therapy can be an anti-PD-L1 antibody listed in Table 1 above (e.g., REGN3504 or atezolizumab) or a functional variant thereof. Such functional variants are substantially similar to the reference antibody (e.g., those listed in Table 1 above), both structurally and functionally. A functional variant comprises substantially the same V_{H} and V_{L} CDRs as the reference antibody. For example, it may comprise only up to 8 (e.g., 8, 7, 6, 5, 4, 3, 2, or 1) amino acid residue variations in the total CDR regions of the antibody and binds the same epitope of a checkpoint molecule (e.g., CTLA-4, PD-1, or PD-L1) with substantially similar affinity (e.g., having a K_{D} value in the same order). In some instances, the functional variants may have the same heavy chain CDR3 as the reference antibody, and optionally the same light chain CDR3 as the exemplary antibody. Alternatively or in addition, the functional variants may have the same heavy chain CDR2 as the reference antibody. Such a functional variant may comprise a V_{H} fragment having CDR amino acid residue variations in only the heavy chain CDR1 as compared with the V_{H} of the reference antibody. In some examples, the antibody may further comprise a V_{L} fragment having the same V_{L} CDR3, and optionally same V_{L} CDR1 or VL CDR₂ as the reference antibody.

Alternatively or in addition, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

(iii) Radiotherapy

In some embodiments, any of the combined cancer therapies disclosed herein may comprise a radiotherapy. Radiotherapy (also known as radiation therapy) is a therapy that uses ionizing radiation to control or kill cancer cells.

Radiotherapy may comprise external, internal, brachytherapy, or systemic exposure, e.g., with a radionuclide (e.g., a β-emitting radionuclide, an α-emitting radionuclide, or a γ-ray emitting radionuclide), electron capturing radionuclides, antibody radionuclide conjugate, or another targeted radionuclide conjugate.

Exemplary β-emitting radionuclide includes, but are not limited to, ³²Phosphorus, ⁶⁷Copper, ⁷⁷Bromine, ⁸⁹Strontiuim, ⁹⁰Yttrium, ¹⁰⁵Rhodium, ¹³¹Iodine, ¹³⁷Cesium, ¹⁴⁹Prometheum, ¹⁵³Samarium, ¹⁶⁶Holmium, ¹⁷⁷Lutetium, ¹⁸⁶Rhenium, ¹⁸⁸Rhenium, or ¹⁹⁹Gold. Exemplar α-emitting radionuclides include, but are not limited to, ²¹¹Astatine, ²¹³Bismuth, ²²³Radium, ²²⁵Actinium, or ²²⁷Thorium. Exemplary γ-ray emitting radionuclides include ¹⁹²Iridium.

Exemplary electron capturing radionuclides include, but are not limited to, ⁶⁷Gallium, ¹⁰³Palladium, or ¹²⁵Iodine.

Exemplary antibody radionuclide conjugates include, but are not limited to, ⁹⁰Y-ibritumomab tiuxetane, ¹³¹I-tositumomab, ²²⁵Ac-lintuzumab satetraxetan, ²²⁷Th-anetumab corixetan, ⁹⁰Y-epitumomab cituxetan, ⁹⁰Y-clivatuzumab tetraxetan, ¹⁷⁷Lu-lilotomab satetraxetan, ⁹⁰Y-rosopatamab tetraxetan, ⁹⁰Y-tabituximab barzuxetan, or ⁹⁰Y-tacatuzumab tetraxetan. Additional targeted radionuclide conjugates include ¹³¹I-PSMA, ⁹⁰Y-PSMA, ¹⁷⁷Lu-PSMA, or ¹⁷⁷Lu-satoreotide tetraxetan. In some instances, an antibody radionuclide conjugate can be used in the combined cancer therapy.

In some embodiments, radiation therapy disclosed herein may includes external beam radiation therapy with X-rays (photons), gamma rays from ⁶⁰Cobalt or other radioactive isotopes, neutrons, electrons, protons, carbon ions, helium ions, and other charged particles. Radiation therapy also includes brachytherapy and radio-pharmaceuticals that emits gamma rays, alpha particles, beta particles, Auger electrons, or other types of radioactive particles from isotopes including ³²Phosphorus, ⁶⁷Copper, ⁷⁷Bromine, ⁸⁹Strontium, ⁹⁰Yttrium, ¹⁰⁵Rhodium, ¹³¹Iodine, ¹³⁷Cesium, ¹⁴⁹Prometheum, ¹⁵³Samarium, ¹⁶⁶Holmium, ¹⁷⁷Lutetium, ¹⁸⁶Rhenium, ¹⁸⁸Rhenium ¹⁹⁹Gold, ²¹¹Astatine, ²¹³Bismuth, ²²³Radium, ²²⁵Actinium, or ²²⁷Thorium, ¹⁹²Iridium, ⁶⁷Gallium, ¹⁰³Palladium, ¹²⁵Iodine, and other radioactive isotopes (e.g., ¹⁹²Iridium, ¹²⁵Iodine, ¹³⁷Cesium, ¹⁰³Palladium, ³²Phosphorus, ⁹⁰Yttrium, ⁶⁷Gallium, ²¹¹Astatine, or ²²³Radium). Radiation therapy also includes radioimmunotherapy (RIT) with antibodies or small molecules that are conjugated to radioactive isotopes including ¹³¹Iodine, ⁹⁰Yttrium, ²²⁵Actinium, ²¹¹Astatine, ⁶⁷Gallium, ¹⁷⁷Lutetium, ²²⁷Thorium, and other radioactive isotopes.

### B. Combined Cancer Therapy

The combined cancer therapy disclosed herein comprises at least one compound of Formula (I) disclosed herein (e.g., Compound 568, 569, 570 or 574) or a pharmaceutically acceptable salt thereof (e.g., a mesylate salt), at least one immune checkpoint inhibitor, and optionally a radiotherapy. In some instances, at least two of therapeutic agents (e.g., all of the three therapeutic agents) can be administered to a subject in sequential manners. In some instances, at least two of the therapeutic agents (e.g., all of the three therapeutic agents) may be administered to the subject simultaneously. In some examples, a compound of Formula (I) and radiotherapy may be given to a subject on the same date, and the checkpoint inhibitor may be given to the subject at least one day after administration of the compound and radiotherapy.

### (i) Pharmaceutical Compositions

To perform the cancer therapy disclosed herein, any of the Formula (I) compounds, the immune checkpoint inhibitors, or the radiotherapeutic agents can be formulated in a pharmaceutical composition. A “pharmaceutical composition” refers to a formulation of any of the therapeutic agents disclosed herein and a medium generally accepted in the art for the delivery of the therapeutic agent to mammals, e.g., humans. Such a medium includes all pharmaceutically acceptable carriers, diluents, or excipients therefor. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the combined cancer therapy disclosed herein can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ PLURONICS™ or polyethylene glycol (PEG).

In some examples, the pharmaceutical composition described herein comprises liposomes containing the therapeutic agent disclosed herein (e.g., the Formula (I) compound, or an antibodies or the encoding nucleic acids as disclosed herein), which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The therapeutic agent (e.g., the Formula (I) compound, any of the checkpoint inhibitory antibodies, or the encoding nucleic acids), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### (ii) Cancer Therapy

In some embodiments, provided herein are combined cancer treatment methods comprising one or more Formula (I) compounds (e.g., Compound 568, 569, 570, or 574) or a pharmaceutically acceptable salt thereof (e.g., a mesylate salt), one or more immune checkpoint inhibitors (e.g., a PD1 antagonist such as an anti-PD-1 antibody or an anti-PD-L1 antibody), and optionally one or more radiation agents as those disclosed herein. In some embodiments, provided herein are combined cancer treatment methods comprising one or more Formula (I) compounds (e.g., Compound 568, 569, 570, or 574) or a pharmaceutically acceptable salt thereof (e.g., a mesylate salt), one or more radiation agents as those disclosed herein, and optionally one or more immune checkpoint inhibitors (e.g., a PD1 antagonist such as an anti-PD-1 antibody or an anti-PD-L1 antibody). In specific examples, the combined cancer treatment disclosed herein comprises one or more Formula (I) compounds (e.g., Compound 568, 569, 570, or 574) or a pharmaceutically acceptable salt thereof (e.g., a mesylate salt), one or more immune checkpoint inhibitors (e.g., a PD1 antagonist such as an anti-PD-1 antibody or an anti-PD-L1 antibody), and one or more radiation agents as those disclosed herein.

A Formula (I) compound, when used in any of the combined therapy, may increase the potency of the checkpoint inhibitor and optionally the radiotherapy. In some instances, it allows the dose of the other treatment to be reduced, which may reduce the frequency and/or severity of adverse events associated with the other drug therapy. For example, side effects of radiation (e.g., oral or gastrointestinal mucositis, dermatitis, pneumonitis, or fatigue) may be reduced in patients receiving a combination therapy including a compound of the invention and reduced dose radiotherapy (e.g., incidence of the adverse events may be reduced by at least 1%, 5%, 10%, or 20%) relative to patients receiving standard full dose radiotherapy without a compound of the invention. Additionally, other adverse events that may be reduced in patients receiving a combination therapy including a compound of the invention and reduced dose radiotherapy (e.g., incidence of the adverse events may be reduced by at least 1%, 5%, 10%, or 20%) relative to patients receiving standard full dose radiotherapy without a compound of the invention may be late effects of radiation, e.g., radiation-induced lung fibrosis, cardiac injury, bowel obstruction, nerve injury, vascular injury, lymphedema, brain necrosis, or radiation-induced cancer. Similarly, when the compound is administered in a combination therapy with another anti-cancer drug (e.g., those described herein), the combined therapy may cause the same or even increased tumor cell death, even when the dose of the other anti-cancer drug is lowered. Reduced dosages of other anti-cancer drugs thus may reduce the severity of adverse events caused by the other anti-cancer drugs.

The treatment comprising the Formula (I) compound may be performed to the subject prior to the treatment comprising the immune checkpoint inhibitor. Alternatively, the treatment comprising the Formula (I) compound may be performed to the subject after the treatment comprising the immune checkpoint inhibitor. In other instances, treatment comprising the Formula (I) compound may be performed to the subject concurrently with the treatment comprising the immune checkpoint inhibitor.

When the combined cancer therapy further comprises radiotherapy, the radiotherapy may be performed before, after, or concurrently with the treatment comprising the Formula (I) compound and/or the treatment comprising the immune checkpoint inhibitor.

Accordingly, in some embodiments, the treatment method disclosed herein comprises administering to a subject in need of the treatment an effective amount of the compound of Formula (I), wherein the subject has been treated with the immune checkpoint inhibitor or is currently on an anti-tumor treatment that comprises an immune checkpoint inhibitor. In some embodiments, the treatment method disclosed herein comprises administering to a subject in need of the treatment an effective amount of the immune checkpoint inhibitor, wherein the subject has been treated with the Formula (I) compound or is currently on an anti-tumor treatment that comprises the Formula (I) compound. In some embodiments, the treatment method disclosed herein comprises administering to a subject in need of the treatment (i) an effective amount of the Formula (I) compound, and (ii) an effective amount of the immune checkpoint inhibitor. Any of the methods disclosed herein may further comprise administering to the subject an effective amount of a radiation agent.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for a Formula (I) compound, an immune checkpoint inhibitor, or a radiation agent as described herein may be determined empirically in individuals who have been given one or more administration(s) of the therapeutic agent. Individuals are given incremental dosages of the agonist. To assess efficacy of the agonist, an indicator of the disease/disorder can be followed.

In some embodiments, the Formula (I) compound may be administered to the subject orally once a day or twice a day. When a commercially available checkpoint inhibitor (e.g., those listed in Table 1 above), and optionally a commercially available radiation agent, is to be co-used with the Formula (I) compound, dosage and dosing schedule may follow routine practice. In some instances, the dosage of each therapeutic agent used in the combined therapy may be lower than its dosage for monotherapy.

For the purpose of the present disclosure, the appropriate dosage of a therapeutic agent as described herein will depend on the specific agent (or compositions thereof) employed, the type and severity of the disease/disorder, whether the agent is administered for preventive or therapeutic purposes, previous therapy, the patient's clinical history and response to the agonist, and the discretion of the attending physician. Typically the clinician will administer an antibody, until a dosage is reached that achieves the desired result. In some embodiments, the desired result is an increase in anti-tumor immune response in the tumor microenvironment. Methods of determining whether a dosage resulted in the desired result would be evident to one of skill in the art. Administration of one or more therapeutic agents can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

The subject to be treated by the methods described herein can be a mammal, more preferably a human. Mammals include, but are not limited to, farm animals, sport animals, pets, primates, horses, dogs, cats, mice and rats. The subject to be treated by the methods disclosed herein may be a human patient having, suspected of having, or at risk for an oncological disease (cancer), for example, a premalignant tumor or a malignant tumor. In some instances, the human patient may have a solid tumor or a hematologic cancer.

In some embodiments, the cancer for treatment is a hematologic cancers, for example, leukemia and lymphoma. Non-limiting examples of cancers include acute myelogenous leukemia, acute lymphoblastic leukemia, acute megakaryocytic leukemia, promyelocytic leukemia, erythroleukemia, lymphoblastic T cell leukemia, chronic myelogenous leukemias, chronic lymphocytic leukemia, hairy-cell leukemia, chronic neutrophilic leukemia, plasmacytoma, immunoblastic large cell leukemia, mantle cell leukemia, multiple myelomas, malignant lymphoma, diffuse large B-cell lymphoma, Hodgkin's lymphoma, non-Hodgkin's lymphoma, lymphoblastic T cell lymphoma, Burkitt's lymphoma, and follicular lymphoma.

In some embodiments, the cancer for treatment is a solid tumor. Non-limiting examples of solid tumors include brain cancers (e.g., astrocytoma, glioma, glioblastoma, medulloblastoma, or ependymoma), bladder cancer, breast cancer, central nervous system cancers, cervical cancer, colon cancer, endometrial cancer, esophageal cancer, gastrointestinal stromal tumor, gastric cancer, head and neck cancers, buccal cancer, cancer of the mouth, hepatocellular cancer, lung cancer, melanoma, Merkel cell carcinoma, mesothelioma, nasopharyngeal cancer, neuroblastoma, osteosarcoma, ovarian cancer, pancreatic cancer, prostate cancer, renal cancer, salivary gland cancer, sarcomas, testicular cancer, urothelial cancer, vulvar cancer, and Wilm's tumor. Preferably, the methods of the invention are used in the treatment of lung cancer, head and neck cancer, pancreatic cancer, rectal cancer, glioblastoma, hepatocellular carcinoma, cholangiocarcinoma, metastic liver lesions, melanoma, bone sarcoma, soft tissue sarcoma, endometrial cancer, cervical cancer, prostate cancer, or Merkel cell carcinoma.

In still further embodiments, examples of cancer to be treated using methods disclosed herein but are not limited to metastases and metastatic cancer. For example, the methods and uses disclosed herein for treating cancer may involve treatment of both primary tumors and metastases.

A subject having a target cancer can be identified by routine medical examination, e.g., laboratory tests, organ functional tests, CT scans, or ultrasounds. In some embodiments, the subject to be treated by the method described herein may be a human cancer patient who has undergone or is subjecting to an anti-cancer therapy, for example, chemotherapy, radiotherapy, immunotherapy, or surgery.

A subject suspected of having any of such target disease/disorder might show one or more symptoms of the disease/disorder. A subject at risk for the disease/disorder can be a subject having one or more of the risk factors for that disease/disorder.

Conventional methods, known to those of ordinary skill in the art of medicine, can be used to administer the Formula (I) compound or the pharmaceutically acceptable salt thereof, the immune checkpoint inhibitor, and optionally the radiation agent to the subject, depending upon the type of disease to be treated or the site of the disease. Any of these therapeutic agents can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, any of the therapeutic agents can be administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

The particular dosage regimen, i.e., dose, timing and repetition, used in the method described herein will depend on the particular subject and that subject's medical history.

In some embodiments, a combined therapy disclosed herein may reduce the tumor size in a subject (e.g., a human patient), at least by 10%, 20%, 30%, 40%, 50%, 60%, 70%, 80%, or 90%, or may eliminate the tumor (e.g., relative to the tumor size at the time of the commencement of the therapy or relative to a reference subject that receives placebo instead of the compound of the invention). In some embodiments, methods of the invention may reduce the tumor burden in a subject at least by 10%, 20%, 30%, 40%, 50%, 60%, 70%, 80%, or 90%, or may eliminate the tumor (e.g., relative to the tumor burden at the time of the commencement of the therapy or relative to a reference subject that receives placebo instead of the compound of the invention). In some embodiments, methods of the invention may increase mean survival time of the subject, e.g., by at least 10%, 20%, 30%, 40%, 50%, 60%, 70%, 80%, 90%, 100%, 150%, or 200% (e.g., relative to a reference subject that receives placebo instead of the compound of the invention). In some embodiments, methods of the invention may increase the ability of radiation therapy or drug therapy to palliate pain or other symptoms for a longer mean time for the subject, e.g., by at least 10%, 20%, 30%, 40%, 50%, 60%, 70%, 80%, 90%, 100%, 150%, or 200% (e.g., relative to a reference subject that receives placebo instead of the compound of the invention).

Treatment efficacy for a target disease/disorder can be assessed by methods well-known in the art.

### II. Kits for Therapeutic Applications

The present disclosure also provides kits for use in treating or alleviating a target cancer, such as hematologic cancer or a solid tumor as described herein. Such kits can include one or more containers comprising one or more Formula (I) compound as disclosed herein (e.g., Compound 568, 569, 570, or 574) or a pharmaceutically acceptable salt thereof (e.g., a mesylate salt) and one or more containers comprising one or more immune checkpoint inhibitors such as PD1 antagonists (e.g., those listed in Table 1 above). Optionally, the kit may further comprise a container comprising a radiation agent.

In some embodiments, the kit can comprise instructions for use in accordance with any of the methods described herein. The included instructions can comprise a description of administration of the Formula (I) compound and the immune checkpoint inhibitor, and optionally the radiation agent, to treat, delay the onset, or alleviate a target cancer as those described herein. The kit may further comprise a description of selecting an individual suitable for treatment based on identifying whether that individual has the target cancer, e.g., applying a diagnostic method as known in the art. In still other embodiments, the instructions comprise a description of administering the therapeutic agents to an individual at risk of the target cancer.

The instructions relating to the use of the Formula (I) compound, the immune checkpoint inhibitor, and optionally the radiation agent, generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or immune disorders (e.g., an autoimmune disease). Instructions may be provided for practicing any of the methods described herein.

The kits disclosed herein are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-CD19 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (lRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Preparation and Characterization of Exemplary Formula (I) Compounds

Exemplary Formula (I) Compounds 568, 569, 570, or 574 were synthesized as follows. See also the synthetic scheme provided herein.

### Methyl 1-(6-bromo-7-fluoro-3-nitroquinolin-4-yl)cyclobutane-1-carboxylate

This intermediate was synthesized as described below, following the synthetic scheme of:

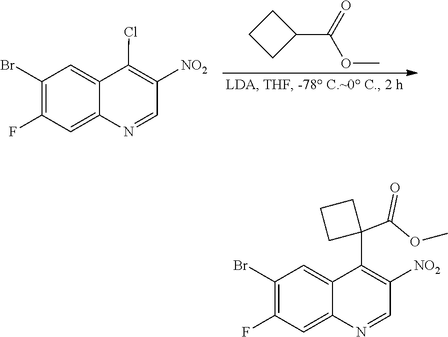

A solution of methyl cyclobutanecarboxylate (0.73 g, 6.38 mmol) in tetrahydrofuran (5.00 mL) was treated with freshly prepared lithium diisopropylamide (6.38 mmol) in tetrahydrofuran (45.0 mL) for 1 hour at −78° C. under nitrogen atmosphere followed by the addition of 6-bromo-4-chloro-7-fluoro-3-nitroquinoline (1.50 g, 4.91 mmol) in portions over 2 min. After stirring for additional 1 hour at ambient temperature, the reaction was quenched by saturated aqueous ammonium chloride (60.0 mL) and diluted with water (120 mL). The resulting mixture was extracted with ethyl acetate (3×60.0 mL). The combined organic layers was washed with brine (2×50.0 mL) and dried over anhydrous sodium sulfate. After filtration, the filtrate was concentrated under reduced pressure. The residue was purified by silica gel column chromatography, eluted with 1%˜2% ethyl acetate in petroleum ether to afford the title compound as a colorless solid (240 mg, 13%): ¹H NMR (400 MHz, CDCl₃) δ 9.14 (s, 1H), 8.20 (d, J=7.2 Hz, 1H), 7.90 (d, J=8.8 Hz, 1H), 3.84 (s, 3H), 3.12-2.99 (m, 1H), 2.58-2.48 (m, 3H), 1.91-1.83 (m, 1H), 1.45-1.27 (m, 1H); MS: [(M+1)]⁺=383.17, 385.17.

### 8′-Bromo-7′-fluorospiro[cyclobutane-1,1′-pyrrolo[2,3-c]quinolin]-2′(3′H)-one

This intermediate was synthesized as described below, following the synthetic scheme of:

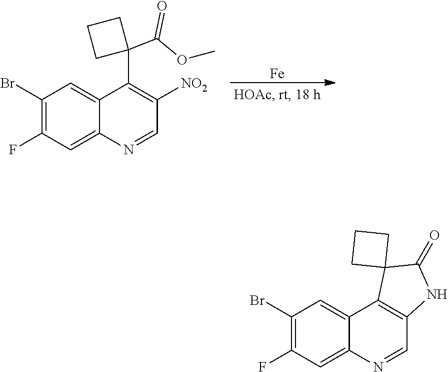

A mixture of methyl 1-(6-bromo-7-fluoro-3-nitroquinolin-4-yl)cyclobutane-1-carboxylate (240 mg, 0.63 mmol) and iron powder (350 mg, 6.26 mmol) in acetic acid (10.0 mL) was stirred for 18 hours at ambient temperature. The resulting mixture was filtered and the filter cake was washed with ethyl acetate (5×100 mL). The filtrate was concentrated under reduced pressure. The residue was purified by silica gel column chromatography, eluted with 1%˜2% methanol in dichloromethane to afford the title compound as a light yellow solid (100 mg, 50%): ¹H NMR (400 MHz, DMSO-d₆) δ 10.75 (s, 1H), 8.68 (s, 1H), 8.52 (d, J=7.5 Hz, 1H), 7.98 (d, J=10.1 Hz, 1H), 2.90-2.75 (m, 2H), 2.50-2.37 (m, 4H); MS: [(M+1)]⁺=321.15, 323.15.

### 8′-Bromo-7′-fluoro-3′-methylspiro[cyclobutane-1,1′-pyrrolo[2,3-c]quinolin]-2′(3′H)-one

This intermediate was synthesized as described below, following the synthetic scheme of:

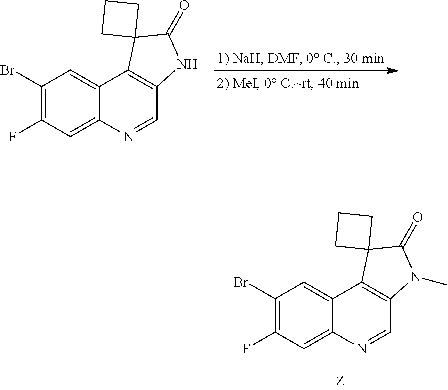 [SMILES: O=C1Nc2cnc3cc(F)c(Br)cc3c2C12CCC2 ; CN1C(=O)C2(CCC2)c2c1cnc1cc(F)c(Br)cc21]

A solution of 8-bromo-7-fluoro-2,3-dihydrospiro[cyclobutane-1,1-pyrrolo[2,3-c]quinoline]-2-one (100 mg, 0.31 mmol) in N,N-dimethylformamide (10.0 mL) was treated with sodium hydride (19.9 mg, 0.50 mmol, 60% dispersed in mineral oil) at 0° C. for 30 min under nitrogen atmosphere followed by the addition of iodomethane (66.3 mg, 0.47 mmol). After stirring for additional 40 min at ambient temperature, the reaction was quenched by saturated aqueous ammonium chloride (10.0 mL). The resulting mixture was diluted with water (100 mL) and extracted with ethyl acetate (3×30.0 mL). The combined organic layers was washed with brine (2×20.0 mL) and dried over anhydrous sodium sulfate. After filtration, the filtrate was concentrated under reduced pressure. The residue was purified by Prep-TLC (DCM/MeOH=20/1, v:v) to afford the title compound as a colorless solid (102 mg, 98%): ¹H NMR (400 MHz, CD₃OD) δ 8.78 (s, 1H), 8.61 (d, J=7.4 Hz, 1H), 7.85 (d, J=9.8 Hz, 1H), 3.36 (s, 3H), 2.94-2.85 (m, 2H), 2.72-2.61 (m, 3H), 2.56-2.48 (m, 1H); MS: [(M+1)]⁺=335.00, 337.00.

### tert-Butyl N-(2-hydroxyethyl)-N-(propan-2-yl)carbamate

This intermediate was synthesized as described below, following the synthetic scheme of:

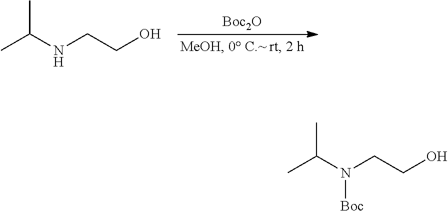

To a solution of 2-[(propan-2-yl)amino]ethan-1-ol (40.0 g, 388 mmol) in methanol (300 mL) was added di-tert-butyl dicarbonate (127 g, 586 mmol) dropwise at 0° C. The resulting mixture was stirred for 2 hours at ambient temperature and concentrated under reduced pressure. The residue was purified by silica gel column chromatography, eluted with 0%˜4% ethyl acetate in petroleum ether. The desired fractions were collected and concentrated under reduced pressure to afford the title compound as a colorless oil (65.0 g, 82%): ¹H NMR (400 MHz, CDCl₃) δ 4.17 (s, 1H), 3.71 (t, J=5.4 Hz, 2H), 3.30 (t, J=5.4 Hz, 2H), 1.47 (s, 9H), 1.12 (d, J=6.8 Hz, 6H).

### tert-Butyl N-[2-[(5-bromo-3-nitropyridin-2-yl)oxy]ethyl]-N-(propan-2-yl)carbamate

This intermediate was synthesized as described below, following the synthetic scheme of:

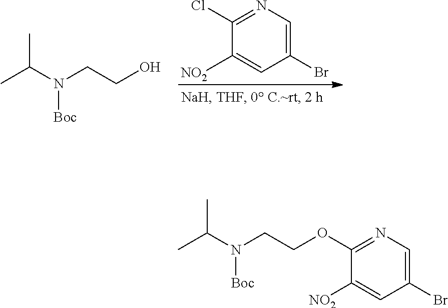 [SMILES: CC(C)N(CCO)C(=O)OC(C)(C)C ; O=[N+]([O-])c1cc(Br)cnc1Cl ; CC(C)N(CCOc1ncc(Br)cc1[N+](=O)[O-])C(=O)OC(C)(C)C]

A solution of tert-butyl N-(2-hydroxyethyl)-N-(propan-2-yl)carbamate (15.4 g, 75.8 mmol) in anhydrous tetrahydrofuran (250 mL) was treated with sodium hydride (3.30 g, 82.1 mmol, 60% w/w dispersed in mineral oil) for 1 hour at 0° C. under nitrogen atmosphere followed by the addition of 5-bromo-2-chloro-3-nitropyridine (15.0 g, 63.2 mmol) over 2 min at 0° C. After additional 2 hours at 25° C., the reaction was quenched by saturated aqueous ammonium chloride (50.0 mL) and diluted with water (500 mL). The aqueous layer was extracted with ethyl acetate (3×150 mL). The combined organic layers was washed with brine (2×100 mL) and dried over anhydrous sodium sulfate. After filtration, the filtrate was concentrated under reduced pressure. The residue was purified by silica gel column chromatography, eluted with 1%˜18% ethyl acetate in petroleum ether. The desired fractions were collected and concentrated under reduced pressure to afford the title compound as a light yellow oil (18.0 g, 71%): ¹H NMR (400 MHz, CDCl₃) δ 8.42 (d, J=2.4 Hz, 1H), 8.37 (d, J=2.4 Hz, 1H), 4.57 (t, J=6.3 Hz, 2H), 4.32 (s, 1H), 3.51 (t, J=6.3 Hz, 2H), 1.47 (s, 9H), 1.15 (d, J=6.9 Hz, 6H); MS: [(M+1)]⁺=404.00, 406.00.

### tert-Butyl N-[2-[(3-amino-5-bromopyridin-2-yl)oxy]ethyl]-N-(propan-2-yl)carbamate

This intermediate was synthesized as described below, following the synthetic scheme of:

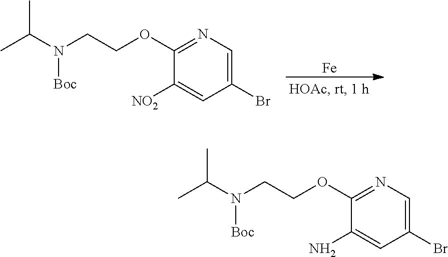

To a solution of tert-butyl N-[2-[(5-bromo-3-nitropyridin-2-yl)oxy]ethyl]-N-(propan-2-yl)carbamate (15.0 g, 37.1 mmol) in acetic acid (150 mL) was added iron powder (20.7 g, 371 mmol) at ambient temperature. After stirring for additional 1 hour at ambient temperature, the resulting mixture was filtered and the filtered cake was washed with tetrahydrofuran (4×100 mL). The filtrate was concentrated under reduced pressure. The residue was purified by silica gel column chromatography, eluted with 20% ethyl acetate in petroleum ether. The desired fractions were collected and concentrated under reduced pressure to afford the title compound as a colorless solid (12.0 g, 86%): ¹H NMR (400 MHz, CD₃OD) δ 7.40 (d, J=2.2 Hz, 1H), 7.04 (d, J=2.2 Hz, 1H), 4.40 (t, J=6.3 Hz, 2H), 4.25-3.99 (m, 1H), 3.52 (t, J=6.3 Hz, 2H), 1.46 (s, 9H), 1.17 (d, J=6.8 Hz, 6H); MS: [(M+1)]⁺=374.10, 376.10.

### tert-Butyl (2-((5-bromo-3-(methylsulfonamido)pyridin-2-yl)oxy)ethyl)(isopropyl)carbamate

This intermediate was synthesized as described below, following the synthetic scheme of:

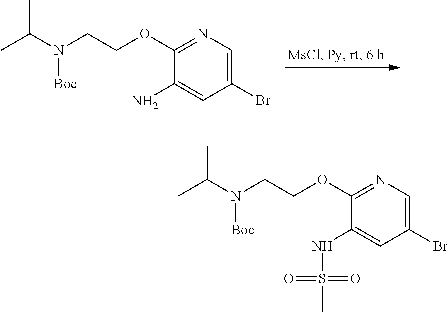

To a solution of tert-butyl N-[2-[(3-amino-5-bromopyridin-2-yl)oxy]ethyl]-N-(propan-2-yl)carbamate (18.8 g, 50.3 mmol) in pyridine (400 mL) was added dropwse methanesulfonyl chloride (8.63 g, 75.4 mmol) at ambient temperature. After stirring for 6 hours at ambient temperature, the resulting mixture was concentrated under reduced pressure. The residue was purified by silica gel column chromatography, eluted with 3%˜25% ethyl acetate in petroleum ether. The desired fractions were collected and concentrated under reduced pressure to afford the title compound as an off-colorless solid (16.3 g, 72%): ¹H NMR (400 MHz, DMSO-d₆) δ 9.41 (s, 1H), 8.05 (d, J=2.2 Hz, 1H), 7.79 (d, J=2.2 Hz, 1H), 4.35 (t, J=6.3 Hz, 2H), 4.15 (s, 1H), 3.43 (t, J=6.3 Hz, 2H), 3.11 (s, 3H), 1.39 (s, 9H), 1.09 (d, J=6.8 Hz, 6H); MS: [(M+1)]⁺=452.00, 454.00.

### tert-Butyl (2-((5-bromo-3-(ethylsulfonamido)pyridin-2-yl)oxy)ethyl)(isopropyl)carbamate

This intermediate was synthesized as described below, following the synthetic scheme of:

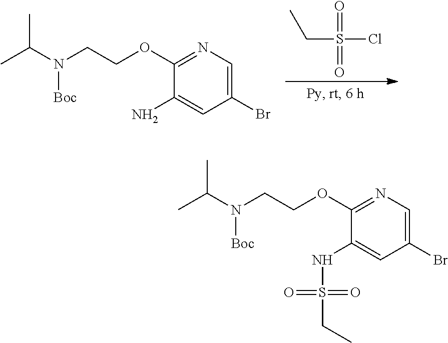

To a stirred solution of tert-butyl (2-((3-amino-5-bromopyridin-2-yl)oxy)ethyl)(isopropyl)carbamate (5.00 g, 13.4 mmol) in pyridine (120 mL) was added ethanesulfonyl chloride (5.15 g, 40.1 mmol) dropwise at ambient temperature under nitrogen atmosphere. After stirring for 6 hours at ambient temperature under nitrogen atmosphere, the resulting mixture was concentrated under reduced pressure. The residue was purified by silica gel column chromatography, eluted with 3%˜25% ethyl acetate in petroleum ether. The desired fractions were collected and concentrated under reduced pressure to afford the title compound as a light yellow solid (3.78 g, 61%): ¹H NMR (400 MHz, CD₃OD) δ 7.92 (s, 1H), 7.87 (s, 1H), 4.48 (t, J=6.6 Hz, 2H), 4.25-4.20 (m, 1H), 3.54 (t, J=6.6 Hz, 2H), 3.13 (t, J=7.3 Hz, 2H), 1.49 (s, 9H), 1.35 (t, J=7.4 Hz, 3H), 1.19 (d, J=6.8 Hz, 6H); MS: [(M+1)]⁺=466.10, 468.10.

### tert-Butyl N-[2-[(5-bromo-3-iodopyridin-2-yl)oxy]ethyl]-N-isopropylcarbamate

This intermediate was synthesized as described below, following the synthetic scheme of:

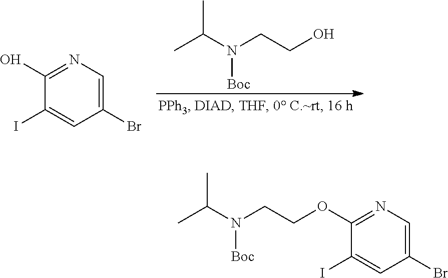 [SMILES: Oc1ncc(Br)cc1I ; CC(C)N(CCO)C(=O)OC(C)(C)C ; CC(C)N(CCOc1ncc(Br)cc1I)C(=O)OC(C)(C)C]

To a solution of 5-bromo-3-iodopyridin-2-ol (10.0 g, 33.3 mmol) in anhydrous tetrahydrofuran (300 mL) were added triphenylphosphine (11.4 g, 43.3 mmol), tert-butyl N-(2-hydroxyethyl)-N-isopropylcarbamate (8.80 g, 43.3 mmol) and diisopropyl azodiformate (8.80 g, 43.4 mmol) dropwise at 0° C. The resulting mixture was stirred for additional 16 hours at ambient temperature under nitrogen atmosphere. The resulting mixture was concentrated under reduced pressure. The residue was purified by silica gel column chromatography, eluted with 2%˜10% ethyl acetate in petroleum ether. The desired fractions were collected and concentrated under reduced pressure to afford the title compound as a colorless oil (14.0 g, 87%): ¹H NMR (400 MHz, CDCl₃) δ 8.14 (s, 2H), 4.43 (t, J=6.4 Hz, 2H), 4.38-3.96 (m, 1H), 3.50 (t, J=6.4 Hz, 2H), 1.49 (s, 9H), 1.19 (d, J=6.8 Hz, 6H); MS: [(M+1)]⁺=484.95, 486.95.

### tert-Butyl (2-((5-bromo-3-((1-methylethyl)sulfonamido)pyridin-2-yl)oxy)ethyl)(isopropyl)carbamate

This intermediate was synthesized as described below, following the synthetic scheme of:

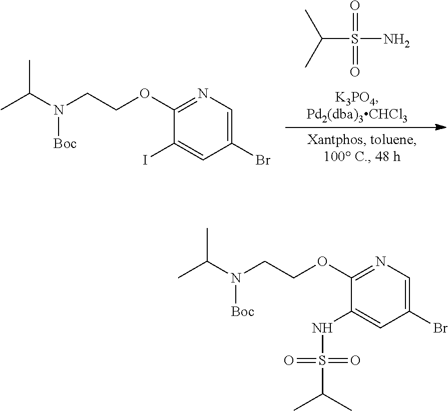

To a mixture of tert-butyl (2-((5-bromo-3-iodopyridin-2-yl)oxy)ethyl)(isopropyl)carbamate (5.00 g, 10.3 mmol), 4,5-bis(diphenylphosphino)-9,9-dimethylxanthene (1.80 g, 3.10 mmol) and propane-2-sulfonamide (1.50 g, 12.4 mmol) in toluene (125 mL) were added tripotassium phosphate (10.9 g, 51.5 mmol) and tris(dibenzylideneacetone)dipalladium-chloroform adduct (1.10 g, 1.10 mmol) at ambient temperature. The resulting mixture was stirred for 48 hours at 100° C. under argon atmosphere. After cooling down to ambient temperature, the resulting mixture was filtered. The filtered cake was washed with ethyl acetate (3×20 mL). The filtrate was concentrated under reduced pressure. The residue was purified by silica gel column chromatography, eluted with 1%˜20% ethyl acetate in petroleum ether. The desired fractions were collected and concentrated under reduced pressure to afford the title compound as a colorless solid (1.90 g, 39%): ¹H NMR (400 MHz, CDCl₃) δ 7.94 (d, J=2.1 Hz, 1H), 7.89 (d, J=2.2 Hz, 1H), 4.44 (t, J=6.3 Hz, 2H), 4.11 (s, 1H), 3.48 (t, J=6.3 Hz, 2H), 3.24-3.30 (m, 1H), 1.48 (s, 9H), 1.41 (d, J=6.8 Hz, 6H), 1.14 (d, J=6.8 Hz, 6H); MS: [(M+1)]⁺=480.20, 482.20.

### N-(5-Bromo-2-[2-[(propan-2-yl)amino]ethoxy]pyridin-3-yl)methanesulfonamide

This intermediate was synthesized as described below, following the synthetic scheme of:

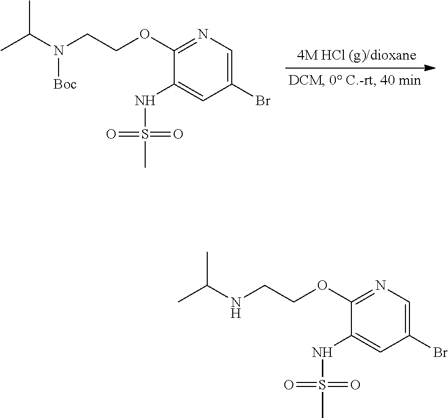 [SMILES: CC(C)N(CCOc1ncc(Br)cc1NS(C)(=O)=O)C(=O)OC(C)(C)C ; CC(C)NCCOc1ncc(Br)cc1NS(C)(=O)=O]

To a solution of tert-butyl N-[2-[(5-bromo-3-methanesulfonamidopyridin-2-yl)oxy]ethyl]-N-(propan-2-yl)carbamate (3.00 g, 6.63 mmol) in dichloromethane (5.00 mL) was treated with hydrogen chloride (20.0 mL, 4 M in 1,4-dioxane) for 40 min at ambient temperature. The resulting mixture was concentrated under reduced pressure. The residue was basified to pH=8 with saturated aqueous sodium bicarbonate (30.0 mL). The resulting mixture was extracted with ethyl acetate (6×200 mL). The combined organic layers was dried over anhydrous sodium sulfate and filtered. The filtrate was concentrated under reduced pressure. The residue was purified by silica gel column chromatography, eluted with 1%˜10% methanol in dichloromethane. The desired fractions were collected and concentrated under reduced pressure to afford the title compound as a colorless solid (1.20 g, 50%): ¹H NMR (400 MHz, DMSO-d₆) δ 7.68 (d, J=2.3 Hz, 1H), 7.61 (d, J=2.3 Hz, 1H), 5.75 (s, 1H), 4.36 (t, J=5.2 Hz, 2H), 3.19-3.12 (m, 1H), 3.07 (t, J=5.1 Hz, 2H), 2.84 (s, 3H), 1.15 (d, J=6.4 Hz, 6H); MS: [(M+1)]⁺=352.10, 354.10.

### N-(5-(7′-Fluoro-3′-methyl-2′-oxo-2′,3′-dihydrospiro[cyclobutane-1,1′-pyrrolo[2,3-c]quinolin]-8′-yl)-2-(2-(isopropylamino)ethoxy)pyridin-3-yl)propane-2-sulfonamide (compound 568)

Compound 568 was synthesized as described below, following the synthetic scheme of:

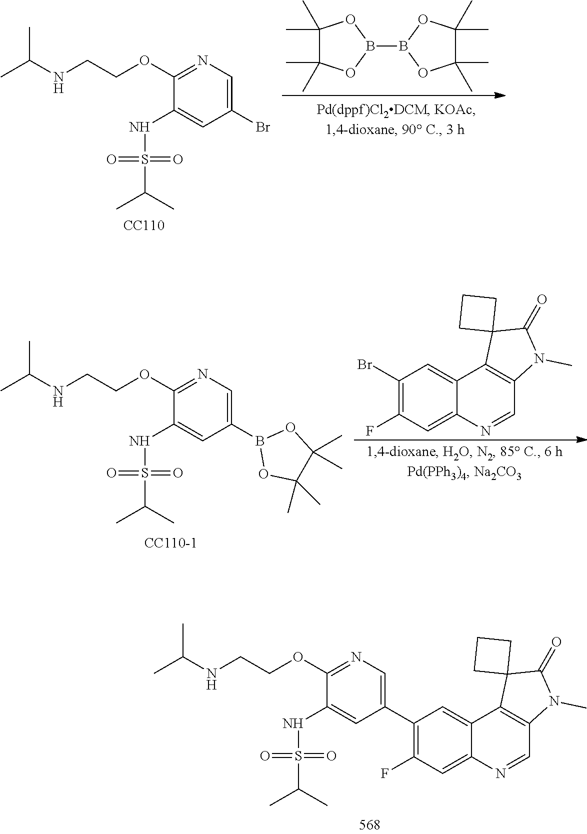

To a solution of N-[5-bromo-2-[2-(isopropylamino)ethoxy]pyridin-3-yl]propane-2-sulfonamide (2.00 g, 5.26 mmol) in 1,4-dioxane (50.0 mL) were added bis(pinacolato)diboron (4.01 g, 15.8 mmol), potassium acetate (2.06 g, 21.1 mmol) and bis(diphenylphosphino)ferrocene]dichloro palladium (II) dichloromethane adduct (0.34 g, 0.42 mmol) at ambient temperature. The resulting mixture was stirred for 3 hours at 90° C. under nitrogen atmosphere. The resulting mixture was cooled down to ambient temperature followed by the additions of 8-bromo-7-fluoro-3-methylspiro[cyclobutane-1,1-pyrrolo[2,3-c]quinolin]-2-one (1.26 g, 3.76 mmol), water (12.5 mL), sodium carbonate (0.80 g, 7.55 mmol) and tetrakis(triphenylphosphine)palladium (0) (0.43 g, 0.38 mmol). The resulting mixture was stirred for 6 hours at 85° C. under nitrogen atmosphere. After cooling down to ambient temperature, the resulting mixture was concentrated under reduced pressure. The residue was purified by reversed phase flash chromatography with the following conditions: Column: Spherical C18, 20˜40 μm, 120 g; Mobile phase A: water (plus 10 mM NH₄HCO₃); Mobile phase B: acetonitrile; Flow rate: 45 mL/min; Gradient (B %): 5%, 2 min; 5%˜25%, 8 min; 25%˜39%, 9 min; 39%, 10 min; 39%˜95%; 3 min; 95%, 2 min; Detector: UV 254 nm; Rt: 21 min. The fractions containing desired product were collected and concentrated under reduced pressure to afford the title compound as a colorless solid (1.66 g, 80%): ¹H NMR (400 MHz, DMSO-d₆) δ 8.88 (s, 1H), 8.35-8.31 (m, 2H), 8.07 (s, 1H), 7.99 (s, 1H), 7.96 (s, 1H), 4.55 (br, 1H), 4.44 (t, J=5.2 Hz, 2H), 3.35-3.30 (m, 4H), 2.93-2.79 (m, 5H), 2.55-2.38 (m, 4H), 1.29 (d, J=7.2 Hz, 6H), 1.02 (d, J=6.0 Hz, 6H); MS: [(M+1)]⁺=556.30.

### N-(5-(7′-Fluoro-3′-methyl-2′-oxo-2′,3′-dihydrospiro[cyclobutane-1,1′-pyrrolo[2,3-c]quinolin]-8′-yl)-2-(2-(isopropylamino)ethoxy)pyridin-3-yl)propane-2-sulfonamide hydrogen chloride

The hydrochloride salt of compound 568 was synthesized as described below, following the synthetic scheme of:

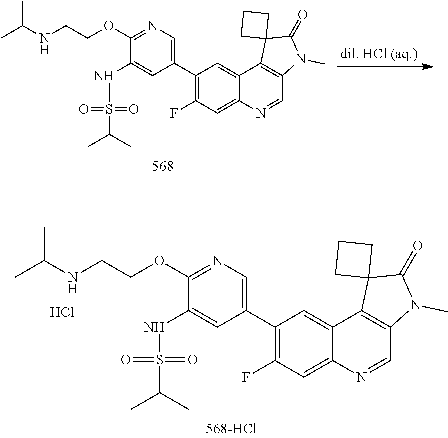

A solution of N-(5-[7-Fluoro-3-methyl-2-oxospiro[cyclobutane-1,1-pyrrolo[2,3-c]quinolin]-8-yl]-2-[2-(isopropylamino)ethoxy]pyridin-3-yl)propane-2-sulfonamide (1.66 g, 2.99 mmol) in diluted aqueous hydrochloric acid solution (392 mL, 3.14 mmol, 0.008 M) and acetonitrile (79.0 mL) was lyophilized to afford the title compound as a yellow solid (1.76 g, 100%): ¹H NMR (400 MHz, DMSO-d₆) δ 9.45 (s, 1H), 9.02 (br, 2H), 8.90 (s, 1H), 8.40 (s, 1H), 8.32 (d, J 8.4 Hz, 1H), 8.16 (s, 1H), 8.00 (d, J 8.4 Hz, 1H), 4.65 (t, J 4.4 Hz, 2H), 3.54-3.40 (m, 3H), 3.31 (s, 3H), 2.91 (t, J=11.2 Hz, 2H), 2.55-2.45 (m, 5H), 1.34-1.30 (i, 12H); MS: [(M+1)]⁺=556.30.

In a similar manner or palladium (II) couples were affected with Intermediates CC108 and CC110 to afford the following compounds example 569 and example 570. See Table 2 below.

TABLE 2
Exemplary Formula (I) Compounds

| Comp. | | | MS: | |
|---|---|---|---|---|
| # | Structure | Name | [(M + 1)]⁺ | ¹H NMR |
| 569 | 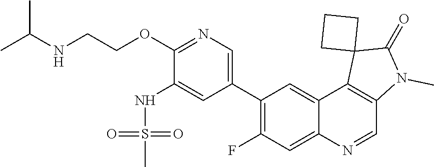 [SMILES: CC(C)NCCOc1ncc(-c2cc3c4c(cnc3cc2F)N(C)C(=O)C42CCC2)cc1NS(C)(=O)=O] | N-(5-(7′-Fluoro-3′- methyl-2′-oxo- 2′,3′- dihydrospiro[cyclo- butane-1,1′- pyrrolo[2,3- c]quinolin]-8′-yl)- 2-(2- (isopropylamino)eth- oxy)pyridin-3- yl)methanesulfona- | 528.30 | ¹H NMR (400 MHz, DMSO-d₆) δ 8.88 (s, 1H), 8.31 (d, J = 8.7 Hz, 2H), 8.01-7.94 (m, 2H), 4.44 (t, J = 5.4 Hz, 2H), 3.31 (s, 3H), 3.05 (s, 3H), 2.97 (t, J = 5.7 Hz, 2H), 2.95-2.85 (m, 3H), 2.60-2.51 (m, 2H), 2.49-2.41 (m, |
| | | mide | | 2H), 1.07 (d, J = 6.3 |
| | | | | Hz, 6H). |
| 569 (HCl salt) | 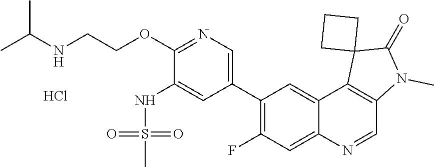 [SMILES: CC(C)NCCOc1ncc(-c2cc3c4c(cnc3cc2F)N(C)C(=O)C42CCC2)cc1NS(C)(=O)=O ; Cl] | N-(5-(7′-Fluoro-3′- methyl-2′-oxo- 2′,3′- dihydrospiro[cyclo- butane-1,1′- pyrrolo[2,3- c]quinolin]-8′-yl)- 2-(2- (isopropylamino)eth- oxy)pyridin-3- yl)methanesulfona- | 528.30 | ¹H NMR (400 MHz, CD₃OD) δ 8.77 (s, 1H), 8.42 (d, J = 8.1 Hz, 1H), 8.18 (t, J = 2.2 Hz, 1H), 8.13 (t, J = 1.8 Hz, 1H), 7.86 (d, J = 12.1 Hz, 1H), 4.63 (t, J = 5.2 Hz, 2H), 3.40-3.32 (m, 6H), 3.04 (s, 3H), 3.00-2.90 (m, 2H), |
| | | mide | | 2.74-2.51 (m, 4H), |
| | | hydrochloride | | 1.31 (d, J = 5.2 Hz, |
| | | | | 6H). |
| 570 | | N-(5-(7′-Fluoro-3′- | 542.30 | ¹H NMR (400 MHz, |
| | | methyl-2′-oxo- | | DMSO-d₆) δ 8.88 (s, |
| | | 2′,3′- | | 1H), 8.35-8.28 (m, |
| | | dihydrospiro[cyclo- | | 2H), 8.02 (t, J = 2.0 |
| | | butane-1,1′- | | Hz, 1H), 7.97 (d, J = |
| | | pyrrolo[2,3- | | 12.2 Hz, 1H), 4.43 (t, |
| | | c]quinolin]-8′-yl)- | | J = 5.4 Hz, 2H), 3.31 |
| | | 2-(2- | | (s, 3H), 3.14 (q, J = |
| | | (isopropylamino)eth- | | 7.2 Hz, 2H), 2.96 (t, J = |
| | | oxy)pyridin-3- | | 5.5 Hz, 2H), 2.93- |
| | | yl)ethanesulfonamide | | 2.82 (m, 3H), 2.60- |
| | | | | 2.53 (m, 2H), 2.49- |
| | | | | 2.40 (m, 2H), 1.28 (t, |
| | | | | J = 7.3 Hz, 3H), 1.05 |
| | | | | (d, J = 6.2 Hz, 6H). |
| 570 | | N-(5-(7′-Fluoro-3′- | 542.30 | ¹H NMR (400 MHz, |
| (HCl | | methyl-2′-oxo- | | CD₃OD) δ 8.82 (s, |
| salt) | | 2′,3′- | | 1H), 8.43 (d, J = 8.0 |
| | | dihydrospiro[cyclo- | | Hz, 1H), 8.35 (d, J = |
| | | butane-1,1′- | | 2.1 Hz, 1H), 8.22 (t, J = |
| | | pyrrolo[2,3- | | 1.7 Hz, 1H), 7.88 |
| | | c]quinolin]-8′-yl)- | | (d, J = 11.8 Hz, 1H), |
| | | 2-(2- | | 4.78 (t, J = 5.1 Hz, |
| | | (isopropylamino)eth- | | 2H), 3.60-3.50 (m, |
| | | oxy)pyridin-3- | | 3H), 3.39 (s, 3H), |
| | | yl)ethanesulfonamide | | 3.27 (t, J = 7.3 Hz, |
| | | hydrochlorie | | 2H), 3.02-2.89 (m, |
| | | | | 2H), 2.76-2.49 (m, |
| | | | | 4H), 1.45-1.37 (m, |
| | | | | 9H). |
| 574 (HCl salt) | 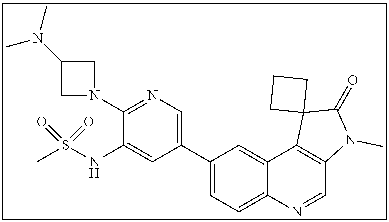 | N-(2-(3- (Dimethylamino)a- zetidin-1-yl)-5-(3′- methyl-2′-oxo- 2′,3′- dihydrospiro[cyclo- butane-1,1′- pyrrolo[2,3- c]quinolin]-8′- yl)pyridin-3- yl)methanesulfona- mide hydrochloride | 507.20 | ¹H NMR (40 0MHz, DMSO-d₆) δ 9.07 (s, 1H), 8.80 (s, 1H), 8.58 (s, 1H), 8.33 (s, 1H), 8.13 (d, J = 8.9 Hz, 1H), 7.95 (d, J = 9.2 Hz, 1H), 7.91 (d, J = 2.1 Hz, 1H), 4.28- 4.21 (m, 2H), 4.02- 3.95 (m, 2H), 3.30 (s, 3H), 3.14 (s, 4H), 2.98-2.90 (m, 2H), 2.60-2.52 (m, 4H), 2.13 (s, 6H) |

Exemplary Formula (I) Compound 574 was synthesized as follows. See also the synthetic scheme provided herein. Its MS: [(M+1)]+ and 1H NMR features are also provided in Table 2 above.

### 1-(5-Bromo-3-nitropyridin-2-yl)-N,N-dimethylazetidin-3-amine

This intermediate was synthesized as described below, following the synthetic scheme of:

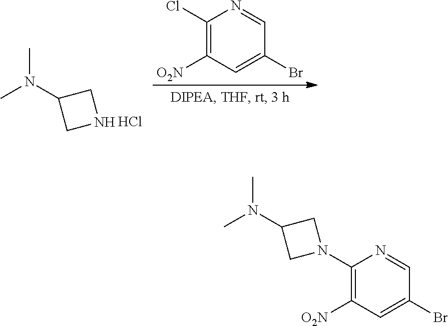

To a stirred solution of N,N-dimethylazetidin-3-amine hydrochloride (0.27 g, 2.08 mmol) and 5-bromo-2-chloro-3-nitropyridine (0.49 g, 2.08 mmol) in tetrahydrofuran (40.0 mL) was added diisopropylethylamine (0.67 g, 5.19 mmol) at ambient temperature. The resulting mixture was stirred for 3 hours and concentrated under reduced pressure. The residue was purified by silica gel column chromatography, eluted with 3%˜9% ethyl acetate in petroleum ether. The desired fractions were collected and concentrated under reduced pressure to afford the title compound as a yellow solid (0.50 g, 59%): ¹H NMR (400 MHz, CDCl₃) δ 8.37 (d, J=2.2 Hz, 1H), 8.29 (d, J=2.2 Hz, 1H), 4.20 (ddd, J=10.0, 7.0, 1.2 Hz, 2H), 3.93 (ddd, J=9.9, 5.1, 1.2 Hz, 2H), 3.16 (tt, J=7.0, 5.1 Hz, 1H), 2.20 (s, 6H); MS: [(M+1)]⁺=301.00, 303.00.

### 1-(5-Bromo-3-nitropyridin-2-yl)-N,N-dimethylazetidin-3-amine

This intermediate was synthesized as described below, following the synthetic scheme of:

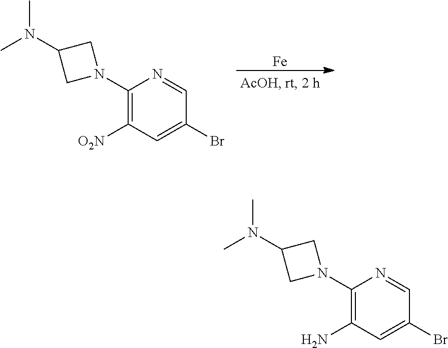

To a solution of 1-(5-bromo-3-nitropyridin-2-yl)-N,N-dimethylpiperidin-4-amine (6.20 g, 20.7 mmol) in acetic acid (90.0 mL) was added iron powder (11.5 g, 206 mmol) at ambient temperature. The resulting mixture was stirred for 2 hours at ambient temperature. The resulting mixture was filtered and the filtered cake was washed with tetrahydrofurane (3×100 mL). The filtrate was concentrated under reduced pressure. The residue was taken up with saturated aqueous sodium carbonate (100 mL) and extracted with ethyl acetate (3×100 mL). The combined organic layers was washed with brine (3×100 mL) and dried over anhydrous sodium sulfate. After filtration, the filtrate was concentrated under reduced pressure. The residue was purified by silica gel column chromatography, eluted with 2%˜4% methanol in dichloromethane to afford the title compound as a grey solid (5.20 g, 92%): ¹H NMR (400 MHz, CDCl₃) δ 7.74 (d, J=2.0 Hz, 1H), 6.93 (d, J=2.0 Hz, 1H), 4.19-4.04 (m, 2H), 3.93-3.88 (m, 2H), 3.21-3.14 (m, 1H), 2.24 (s, 6H); MS: [(M+1)]⁺=271.00, 273.00.

### N-(5-Bromo-2-(3-(dimethylamino)azetidin-1-yl)pyridin-3-yl)methanesulfonamide

This intermediate was synthesized as described below, following the synthetic scheme of:

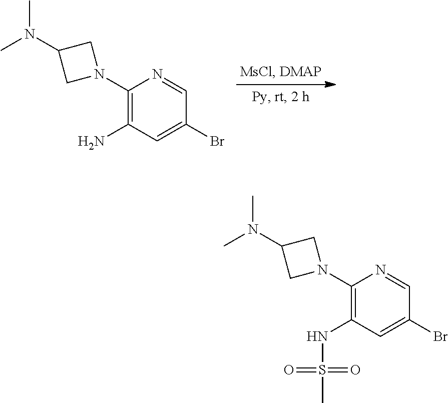 [SMILES: CN(C)C1CN(c2ncc(Br)cc2N)C1 ; CN(C)C1CN(c2ncc(Br)cc2NS(C)(=O)=O)C1]

To a stirred solution of 5-bromo-2-[3-(dimethylamino)azetidin-1-yl]pyridin-3-amine (2.10 g, 7.77 mmol) and N,N-4-dimethylaminopyridine (77.0 mg, 0.63 mmol) in pyridine (70.0 mL) was added methanesulfonyl chloride (1.77 g, 15.5 mmol) dropwise at ambient temperature. After stirring for 3 hours at ambient temperature under nitrogen atmosphere, the resulting mixture was concentrated under reduced pressure. The residue was purified by reversed phase flash chromatography with the following conditions: Column: Spherical C18, 20˜40 μm, 330 g; Mobile Phase A: Water (plus 10 mM NH₄HCO₃), Mobile Phase B: acetonitrile; Flow rate: 65 mL/min; Gradient (B %): 5%˜20%, 8 min; 20%˜40%, 20 min; 40%˜95%, 2 min; 95%, 5 min; Detector: UV 254 nm. The desired fractions were collected at 19 min and concentrated under reduced pressure to afford the title compound as a colorless solid (1.40 g, 52%): ¹H NMR (400 MHz, CD₃OD) δ 7.92 (d, J=2.2 Hz, 1H), 7.61 (d, J=2.2 Hz, 1H), 4.27 (dd, J=8.8, 7.2 Hz, 2H), 3.96 (dd, J=9.0, 5.6 Hz, 2H), 3.24-3.16 (m, 1H), 3.00 (s, 3H), 2.20 (s, 6H); MS: [(M+1)]⁺=349.00, 351.00.

### N-(2-(3-(Dimethylamino)azetidin-1-yl)-5-(4,4,5,5-tetramethyl-1,3,2-dioxaborolan-2-yl)pyridin-3-yl)methanesulfonamide

This intermediate was synthesized as described below, following the synthetic scheme of:

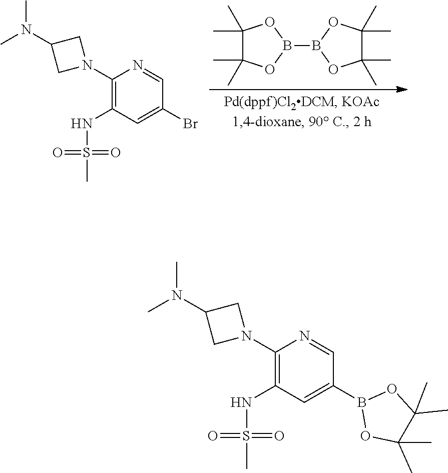

To a solution of N-[5-bromo-2-[3-(dimethylamino)azetidin-1-yl]pyridin-3-yl]methanesulfonamide (1.00 g, 2.86 mmol) and 4 bis(pinacolato)diboron (2.18 g, 8.59 mmol) in 1,4-dioxane (30.0 mL) were added potassium acetate (1.12 g, 11.5 mmol) and bis(diphenylphosphino)ferrocene]dichloro palladium (II) dichloromethane adduct (351 mg, 0.43 mmol) at ambient temperature. The resulting mixture was stirred for 2 hours at 85° C. under nitrogen atmosphere. The resulting mixture was concentrated under reduced pressure. The residue was purified by reversed flash chromatography with the following conditions: Column: Spherical C18, 20˜40 μm, 330 g; Mobile Phase A: Water (plus 10 mM NH₄HCO₃), Mobile Phase B: acetonitrile; Flow rate: 65 mL/min; Gradient (B %): 5%˜20%, 7 min; 20%˜40%, 12 min; 40%˜95%; 2 min; 95%, 5 min; Detector: UV 254 nm. The desired fractions were collected at 20 min and concentrated under reduced pressure to afford the title compound as a yellow solid (800 mg, 71%): ¹H NMR (400 MHz, DMSO-d₆) δ 8.78 (s, 1H), 8.18 (d, J=1.5 Hz, 1H), 7.50 (d, J=1.6 Hz, 1H), 4.19 (t, J=8.0 Hz, 2H), 3.93 (dd, J=8.9, 5.0 Hz, 2H), 3.12-3.04 (m, 1H), 2.99 (s, 3H), 2.09 (s, 6H), 1.27 (s, 12H); MS: [(M+1)]⁺=397.20.

### N-(2-(3-(Dimethylamino)azetidin-1-yl)-5-(3′-methyl-2′-oxo-2′,3′-dihydrospiro[cyclobutane-1,1′-pyrrolo[2,3-c]quinolin]-8′-yl)pyridin-3-yl)methanesulfonamide

This intermediate was synthesized as described below, following the synthetic scheme of:

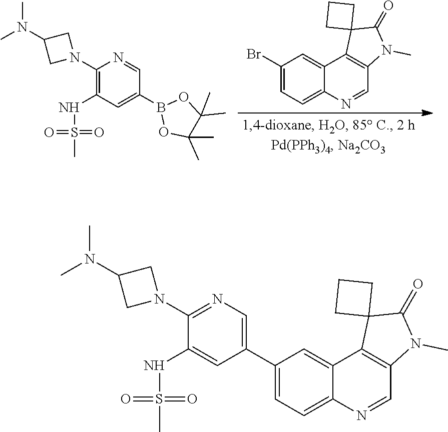

To a solution of 8-bromo-3-methyl-2,3-dihydrospiro[cyclobutane-1,1-pyrrolo[2,3-c]quinolin]-2-one (320 mg, 1.01 mmol) and N-[2-[3-(dimethylamino)azetidin-1-yl]-5-(4,4,5,5-tetramethyl-1,3,2-dioxaborolan-2-yl)pyridin-3-yl]methanesulfonamide (600 mg, 1.51 mmol) in 1,4-dioxane (13.0 mL) and water (2.00 mL) were added sodium carbonate (160 mg, 1.51 mmol) and tetrakis (triphenylphosphine) palladium (0) (175 mg, 0.15 mmol). After stirring for 2 hours at 85° C. under a nitrogen atmosphere, the resulting mixture was concentrated under reduced pressure. The residue was purified by Prep-TLC (DCM/MeOH=8/1, v/v) to afford the title compound as a light yellow solid (350 mg, 69%): ¹H NMR (400 MHz, DMSO-d₆) δ 9.07 (s, 1H), 8.80 (s, 1H), 8.58 (s, 1H), 8.33 (s, 1H), 8.13 (d, J=8.9 Hz, 1H), 7.95 (d, J=9.2 Hz, 1H), 7.91 (d, J=2.1 Hz, 1H), 4.28-4.21 (m, 2H), 4.02-3.95 (m, 2H), 3.30 (s, 3H), 3.14 (s, 4H), 2.98-2.90 (m, 2H), 2.60-2.52 (m, 4H), 2.13 (s, 6H); MS: [(M+1)]⁺=507.20.

### N-(2-(3-(Dimethylamino)azetidin-1-yl)-5-(3′-methyl-2′-oxo-2′,3′-dihydrospiro[cyclobutane-1,1′-pyrrolo[2,3-c]quinolin]-8′-yl)pyridin-3-yl)methanesulfonamide hydrochloride (compound 574)

Compound 574 was synthesized as described below, following the synthetic scheme of:

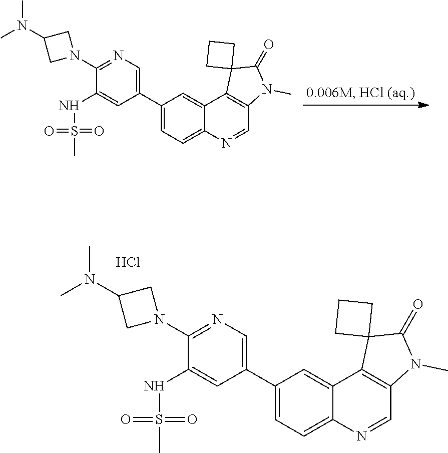

A solution of N-(2-(3-(dimethylamino)azetidin-1-yl)-5-(3′-methyl-2′-oxo-2′,3′-dihydrospiro[cyclobutane-1,1′-pyrrolo[2,3-c]quinolin]-8′-yl)pyridin-3-yl)methanesulfonamide (350 mg, 0.69 mmol) in diluted aqueous hydrochloric acid solution (115 mL, 0.69 mmol, 0.006 M) and acetonitrile (20.0 mL) was lyophilized directly to afford the title compound as an orange solid (375 mg, 100%): ¹H NMR (400 MHz, DMSO-d₆) δ 10.77 (s, 1H), 9.24 (s, 1H), 8.83 (s, 1H), 8.64 (d, J=2.1 Hz, 1H), 8.35 (s, 1H), 8.17 (d, J=8.9 Hz, 1H), 8.02-7.94 (m, 2H), 4.50-4.41 (m, 2H), 4.39-4.32 (m, 2H), 4.27-4.16 (m, 1H), 3.31 (s, 3H), 3.18 (s, 3H), 2.99-2.88 (m, 2H), 2.82 (d, J=4.1 Hz, 6H), 2.63-2.52 (m, 4H); MS: [(M+1)]⁺=507.20.

### Example 2: Target and Off-Target Inhibitory Activities of Exemplary Formula (I) Compounds

Exemplary Formula (I) compounds (568, 569, 570, and 574) were tested for inhibitory activity against target enzymes (ATM and DNA-PK) and off target enzymes (mTOR, PI3Kα/δ, and hERG) following the conditions described below.

### Methods

### ATM In Cell Western Assay:

MCF-7 breast cancer cells were placed in a 384 well plate at the density of 10,000 cells/well (Corning, #356663), 25 μL cells per well. The following day, compounds were added to the plate using a pin tool (Echo 550) to a final concentration of 1 μM through a 3-fold serial dilution (a total of 10 doses). Then, etoposide (Sigma, #E1383) was added to a final concentration of 100 μM. The plate was incubated at 37° C. for 1 h, and the cells were fixed by the addition of 25 μL of fixing solution (8% paraformaldehyde) for 20 minutes at ambient temperature. Cells were permeabilized by 5 washes with 1×PBS (phosphate buffered saline) containing 0.1% Triton X-100; each wash was 5 minutes long. Cells were blocked by adding 50 μL of Odyssey Blocking Buffer (LI-COR, #927-40000) in 384 well plates for 1.5 hours with shaking at ambient temperature. Blocking buffer was then removed, and 20 μL of anti-pKAP1 antibody (Bethyl Laboratories, #A300-767A) (1/2000) solution were added to each well of 384-well plate. The plate was incubated overnight at 4° C. and then washed 5 times with 1×PBST (1×PBS containing 0.1% Tween-20). A secondary antibody (IRDye 800CW Goat anti-Rabbit IgG, LI-COR, #926-32211) (1/5,000) solution containing DNA stain DRAQ5 (CST, #4084L) (1/5,000) (20 μL) was added to each well in the plate, and the plate was incubated 1 hour with gentle shaking in the dark. Cells were washed 5 times with 1×PBST (1×PBS containing 0.1% Tween-20) at ambient temperature with gentle shaking in the dark. After the last wash, the wash solution was removed, the plate was inverted upside down onto a thin paper towel and centrifuged at 1000 rpm for 1 min to absorb all wash buffer. The bottom of the plate was cleaned with a moist, lint-free paper. The plate was immediately scanned using ODYSSEY CLx (LI-COR).

DNA-PK Enzyme-Linked immunosorbent Assay:

On day one, a 96-well plate (ThermoFisher, Cat #: 442404) was coated with GST-p53 (1-101) peptide (purified by Pharmaron, BCS department) by diluting 3 μg of GST-p53 in each well with 0.1 M Na₂CO₃/NaHCO₃ (pH 9.6). The plate was incubated overnight at 4° C. On the second day, the coating buffer was removed, and the plate was washed twice with PBST (1×PBS containing 0.1% Tween-20). The DNA-PK enzyme solution (Invitrogen, #PR9107A; the final DNA-PK concentration: 0.1 μg/mL) was then added. The compounds were serially diluted to the final maximal concentration of 100 nM (3 fold series dilution, a total of 10 doses), and an ATP solution (the final ATP concentration: 20 μM) was added to the plate. Incubate the plate at 25° C. for 1 hour. The plate was washed three times with PBST (1×PBS containing 0.1% Tween-20) and blocked with a solution of PBST and 1% BSA at 4° C. overnight. The third day, the plate was washed four times with PBST (1×PBS containing 0.1% Tween-20). Anti-phospho-p53 primary antibody (cell signaling Technology, #9286, Phospho-p53 (Ser15) (16G8) Mouse mAb) (1/1000) was added to each well. The plate was sealed, incubated 1 h at 37° C., and washed four times with PBST (1×PBS containing 0.1% Tween-20). An HRP-linked secondary antibody (Cell signaling Technology, #7076, Anti-mouse IgG, HRP-linked Antibody) (1/1000) (100 μL) was added to each well. The plate was sealed with tape, incubated 30 min at 37° C., and washed four times with PBST (1×PBS containing 0.1% Tween-20). At this time, 100 μL of TMB (Cell signaling Technology, #7004) substrate were added to each well. The plate was sealed with tape and incubated the plate 10 min at 37° C. Stop solution (Cell signaling Technology, #7002) (100 μL) was added to each well, and the plate was subjected to the absorption detection at 450 nm.

mTOR Biochemical Assay:

mTOR Kinase reactions were performed in a 10 μL volume in low-volume 384-well plates. Typically, PerkinElmer model 6008260 plates were used. The composition of the 1× kinase reaction buffer was: 50 mM HEPES pH 7.5, 0.01% Tween 20, 1 mM EGTA, 10 mM MnCl₂, and 2 mM DTT. The solution of mTOR enzyme (ThermoFisher, #PR8683B; the final mTOR concentration: 0.5 μg/mL) was added, and the compounds were serially diluted to the final maximal concentration of 100 nM (3 fold series dilution, a total of 10 doses). GFP-4E-BP1 (the final concentration: 0.4 μM) and the ATP solution (the final ATP concentration: 3 μM) were added to the 384-well plate. The plate was incubated at 25° C. for 1 hour, and 10 μL of the EDTA solution (20 mM) and Tb-labeled anti-p4E-BP1 antibody (4 nM) in TR-FRET dilution buffer were added to each well. The plate was sealed, incubated 30 min at 25° C., and read on a plate reader configured for LanthaScreen™ TRFRET.

### PI3Kα and PI3Kδ Biochemical Assay:

PI3Kα and PI3Kδ Kinase reactions were performed in a 5 μL volume in low-volume 384-well plates. Typically, PerkinElmer model 6008280 plates were used. The 1× kinase reaction buffer consisted of 50 mM HEPES pH 7.5, 3 mM MgCl₂, 0.03% CHAPS, 1 mM EGTA, 100 mM NaCl, and 2 mM DTT. PI3Kα (ThermoFisher, #PV4788; the final PI3Kα concentration: 120 ng/mL) or PI3Kδ enzyme solution (ThermoFisher, #PV6451; the final PI3Kδ concentration: 250 ng/mL) was added to the plate, compounds were serially diluted to the final maximal concentration of 100 nM (3 fold series dilution, a total of 10 doses), and the PIP2:3PS (the final concentration: 10 μg/mL) and ATP solution (the final ATP concentration: 10 μM) was added to the 384-well plate. The plate was incubated at 25° C. for 1 hour. ADP-Glo reagent buffer (5 μL) was added to each well. The plate was sealed and incubated for 40 min at 25° C. ADP-Glo detection buffer (10 μL) was added to each well, and the plate was incubated for 40 min at 25° C. and read on a plate reader configured for Luminescence.

In-Vitro hERG Inhibition Assay:

The hERG-T-REx™ HEK 293 cells (Invitrogen, K1236) were generated by transfecting the hERG coding sequence in the Tet-regulated expression vector pT-Rex-DEST30 into cells expressing the Tet-repressor (T-Rex™ HEK293), thereby producing cells that can be induced to express high level of hERG channels. The cells were cultured in a medium containing of 85% DMEM, 10% dialyzed FBS, 0.1 mM NEAA, 25 mM HEPES, 100 U/mL Penicillin-Streptomycin, 5 μg/mL Blasticidin, and 400 μg/mL Geneticin. The cells were split using TrypLE™ Express (Gibco, 12604) about three times a week and maintained between ˜40% to ˜80% confluence. Before the assay, the cells were induced with doxycycline (Sigma, D9891) at 1 μg/mL for 48 hours. On the experiment day, the induced cells were resuspended and plated onto the coverslips at 5×10⁵ cells/per 6 cm cell culture dish prior to use. The hERG channel-mediated current was acquired by manual patch clamp recording systems equipped with amplifiers (HEKA, EPC10 and Molecular Devices, multiclamp 700B) and the inverted phase contrast microscope (Olympus, IX51/71/73). Glass pipettes were prepared by micropipette puller (Sutter, P97 and Narishige, PC-10) and qualified by the pipette resistance in the range of 2-4 MOhms. The internal pipette solution was 140 mM KCl, 2 mM MgCl₂, 10 mM EGTA, 5 mM MgATP, and 10 mM HEPES (pH adjusted to 7.35 with KOH), and the external buffer was 132 mM NaCl, 4 mM KCl, 3 mM CaCl₂), 0.5 mM MgCl₂, 11.1 mM glucose, and 10 mM HEPES (pH adjusted to 7.35 with NaOH). The whole-cell configuration was maintained with access resistance continuously monitored (<15 MOhms). The hERG current was elicited by depolarizing membrane to +30 mV for 4.8 sec, and the voltage was set back to −50 mV for 5.2 sec to remove the inactivation and measure the deactivating tail current. The maximum amount of tail current size was used to determine hERG current amplitude. To evaluate the hERG inhibition, the blank vehicle and test articles were perfused to cells under whole-cell recording configuration through the liquid perfusion system (ALA, VM8 gravity-flow delivery system). For dose response assay, test article was applied to the cells accumulatively from low to high concentrations. A positive control (Dofetilide) was used in the experiments to ensure the performance of the cells and operations as major part of method validation. The percentage hERG current inhibition was fitted against dose concentrations to build the dose-response curve and determine IC₅₀.

### Results

Compounds 568, 569, 570 and 574 were investigated in this example. The results are shown in Table 3 below.

TABLE 3
Target and Off-Target Inhibitory Activities of Exemplary Formula (I) Compounds

| | | ATM | DNA- | | PI3K- | |
|---|---|---|---|---|---|---|
| | | IC₅₀ | PK | mTOR | α/δ | hERG |
| | | (nM) | IC₅₀ | IC₅₀ | IC₅₀ | IC₅₀ |
| Compound | nomenclature | (cell) | (nM) | (nM) | (nM) | (μM) |
| 568 | N-(5-(7′-Fluoro-3′-methyl-2′-oxo- | 2.88 | 0.79 | >100 | 18/54 | 14 |
| | 2′,3′-dihydrospiro[cyclobutane-1,1′- | | | | | |
| | pyrrolo[2,3-c]quinolin]-8′-yl)-2-(2- | | | | | |
| | (isopropylamino)ethoxy)pyridin-3- | | | | | |
| | yl)propane-2-sulfonamide | | | | | |
| 569 | N-(5-(7′-Fluoro-3′-methyl-2′-oxo- | 2.43 | 0.63 | >100 | 16/73 | >30 |
| | 2′,3′-dihydrospiro[cyclobutane-1,1′- | | | | | |
| | pyrrolo[2,3-c]quinolin]-8′-yl)-2-(2- | | | | | |
| | (isopropylamino)ethoxy)pyridin-3- | | | | | |
| | yl)methanesulfonamide | | | | | |
| | hydrochloride | | | | | |
| 570 | N-(5-(7′-Fluoro-3′-methyl-2′-oxo- | 3.60 | 0.63 | >100 | 21/55 | 10.5 |
| | 2′,3′-dihydrospiro[cyclobutane-1,1′- | | | | | |
| | pyrrolo[2,3-c]quinolin]-8′-yl)-2-(2- | | | | | |
| | (isopropylamino)ethoxy)pyridin-3- | | | | | |
| | yl)ethanesulfonamide hydrochloride | | | | | |
| 574 | N-(2-(3-(Dimethylamino)azetidin-1- | 1.52 | 1.03 | 19.9 | 0.12/3 | >30 |
| | yl)-5-(3′-methyl-2′-oxo-2′,3′- | | | | | |
| | dihydrospiro[cyclobutane-1,1′- | | | | | |
| | pyrrolo[2,3-c]quinolin]-8′-yl)pyridin- | | | | | |
| | 3-yl)methanesulfonamide | | | | | |
| | hydrochloride | | | | | |

The results show that the exemplary Formula (I) selectively inhibit ATM and DNA-PK but show low or no inhibitory activity against mTOR, PI3Kα/6, and hERG.

### Example 3: Clonogenic and In Vivo Efficacy Assays

Compound 569 was used as an exemplary Formula (I) compound in this example for measuring clonogenic activity and in vivo efficacy.

### Methods

### Human Cancer Cell Culture

Human cancer cell lines, including MCF-7 human breast carcinoma cell line and A549 human lung carcinoma cell line, and HCT116 p53 wild-type and HCT116 p53−/− cell lines were obtained from ATCC. MCF7 and HCT116 cells were cultured in Dulbecco's modified Eagle's medium (DMEM (Gibco #11995-065). MDA-MB-231 human triple negative breast cancer cell line and FADU, human head and neck squamous cell carcinoma line, were obtained from Charles River Laboratories (Morrisville, NC). MDA-MB-231 and A549 cells were cultured in RPMI 1640 medium (Gibco, 11875-093). FADU cells were cultured in Minimum Essential Medium α (MEM α) (Gibco, 12571-063) supplemented with 1 mM Sodium Pyruvate (Gibco, 11360-070), 1×MEM Non-Essential Amino Acids (Gibco, 11140-050). All cell lines were supplemented with 10% fetal bovine serum (FBS) (Corning, 35-010-CV) and 1× Antibiotic-Antimycotic (Gibco #15240-062) and were grown at 37° C. in 5% CO2. All cell lines were authenticated by short tandem repeat profiling and tested negative for mycoplasma.

### Antibodies of ATM and DNA-PKcs

Antibodies recognizing phosphorylated/activated ATM (S1981, #ab81292) and DNA-PKcs (S2056, #ab18192) were purchased from Abcam Biotechnology (Cambridge, MA). Antibodies to ATM (#A1106) and DNA-PKcs (#ab1832) were from Sigma and Abcam, respectively. Phospho-KAP1 (S824, #A300-767A) and KAP1 (A700-014) antibodies were from Bethyl Labs (Montgomery, TX). Phospho-TBK1 (#5483), cGAS (#15102), and pSTING (S366, #19781S) antibodies were from Cell Signaling Technology (CST) (Danvers, MA). STING (PA5-23381) antibodies were from ThermoFisher Scientific (Waltham, MA), and actin (#A5316) antibodies were from Sigma. All antibodies were diluted 1:1000 with the exception of actin which was diluted 1:3000 in 1×TBST (1×TBS-10×TBS, (Corning, 46-012-CM), 0.05% Tween-20 (P7949) Sigma and 1% BSA. Secondary antibodies used were (a) Li-Cor method-Goat anti-Rabbit (926-3221) and Goat anti-Mouse (926-68020) Li-Cor (b) ECL method: Anti-rabbit IgG, HRP-linked (#7074S), CST.

### Immunoblotting Tests

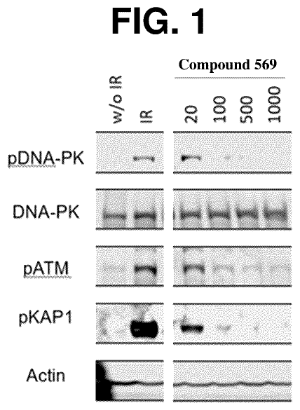

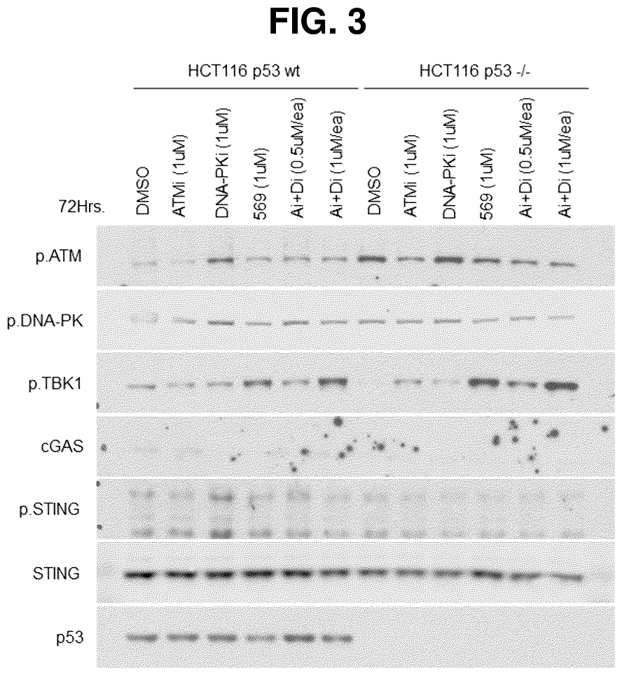

Cells were pretreated with Compound 569 at 20, 100, 500, or 1000 nM or DMSO for 30 min and then irradiated with 0 or 10 Gy (XRAD 160, Precision X Ray) as shown in FIG. 1. In FIG. 3, cells were pretreated with Compound 569 at 1 μM, ATMi (AZD0156) at 1 μM, DNA-PKi (peposertib) at 1 μM as single agents or DMSO or with ATMi and DNA-PKi in combination at 0.5 micromolar each or at 1 micromolar each for 30 min and then irradiated with 0 or 10 Gy (XRAD 160, Precision X Ray) as shown in FIG. 1. One hour after IR, cells were lysed in RIPA buffer (50 nM Tris, pH 8.0, 150 mM NaCl, 0.1% SDS, 0.5% sodium deoxycholate, 1% IGEPAL® CA-630 (Sigma, 18896) for 20 min at 4° C. in the presence of protease and phosphatase inhibitor cocktails (Roche, #04693159001 & #04906837001). Cell lysates were centrifuged at 13,200 g for 10 min at 4° C. and supernatants were analyzed for protein content using BCA assay (Pierce™ BCA Protein Assay Kit, cat #23227, ThermoFisher Waltham, MA 02451) and equal amounts of protein/lane were used for subsequent immunoblot analyses. Proteins were denatured by adding NuPAGE LSD Sample Buffer [4×] (Invitrogen, NP0007) containing β-mercaptoethanol at a final concentration of 2.5% and the samples were boiled for 5 minutes. Samples were loaded into NuPAGE 3-8% Tris-Acetate Protein Gels (Invitrogen, EA03785BOX) and electrophoresed with 1×Tris-Acetate SDS Running Buffer (Invitrogen, LA0041), or were loaded into NuPAGE 4-12% Bis-Tris Protein Gels (Invitrogen, NP0336BOX) and electrophoresed with 1×MOPS SDS Running Buffer (Invitrogen, NP0001).

Proteins were transferred overnight to nitrocellulose membranes (Amersham, 10600003). Membranes were blocked with 5% non-fat dry milk in 1×TBST for 1 hour and incubated with primary antibodies overnight at 4° C. Membranes were washed 4×10 min with 1×TBST at RT, and then incubated with secondary antibodies for 1 hr at RT, followed by washing 3×10 min with 1×TBST at RT. Membranes were visualized with ODYSSEY CLX system, or with ECL (ECL Blotting substrate, Pierce, 32209, 34075, 34095).

### Clonogenic Survival Assay

This assay was performed with Compound 569 as an exemplary Formula (I) compound. Cells were plated in 6-well plates at different densities: 250 cells for no IR control, 5000 cells for 2 Gy of IR and 10000 cells for 4 Gy of IR and cultured overnight. The next day, MCF7 cells were preincubated with compound 569 at 100, 250, 500, or 1000 nM or DMSO for 30 min before being exposed to increasing doses of IR (0, 2, or 4 Gy). The A549 cells were incubated with Compound 569 at 500 or 1000 nM or with DMSO for 30 min before being exposed to increasing doses of IR (0, 2, or 4 Gy). After IR, cells were continuously incubated with DMSO or compound 569 for 5 h before the culture medium was removed and cells were washed with PBS. Cells were cultured in complete medium in the absence of inhibitor for 9 days. Then cells were stained (PBS, 0.0037% v/v formaldehyde, 0.1% crystal violet) rinsed with water and dried.

### Tumor Studies in Murine Models

Female NCr nu/nu mice (Crl:NU(NCr)-Foxn1nu, Strain 490) were obtained from Charles River at 5-6 weeks of age.

FADU or MDA-MB-231 cells were harvested during log phase growth for in vivo implantation. The resuspended cells were washed three times in phosphate buffered saline (PBS) before preparing the working dilution in PBS. FADU cells were diluted to 1×10⁷ cells/mL and MDA-MB-231 cells were diluted to 5×10⁷ cells/mL. Each mouse was injected subcutaneously in the right flank with 100 μL of the cellular suspension. Tumor growth was monitored until the tumors reached a target volume of at least 100 mm³, at which point mice were randomly stratified into four treatment groups.

Following implantation of the tumor cell lines, mice were monitored once weekly for tumor development. Upon detection, tumors were measured in two dimensions using digital calipers. Tumors were treated after reaching at least 100 mm³ and were measured two to three times weekly following enrollment in one of the treatment groups. The study endpoint was defined as tumor quintupling from the volume at time of treatment.

The vehicle used in this study was 0.5% (Hydroxypropyl)methyl cellulose (HPMC) and 0.2% Tween80 dissolved in deionized water. The pH of the vehicle was adjusted to 7.0-8.0 and the vehicle was stored at 4° C. On each day of in vivo treatment an appropriate mass of test compound was added to the vehicle at room temperature. The mixture was vortexed as necessary to generate a final concentration of 1.6 mg/mL.

### Pharmacodynamic (PD) Assays

Tumor studies were performed as follows. Mice bearing FADU or MDA-231 tumors were pre-dosed with vehicle alone or compound 569 alone at 3, 6, and 10 mg/kg. Tumors were then irradiated with 10 Gy 45 min after vehicle or compound 569 administration or left unirradiated (vehicle and compound 569 at 10 mg/kg). Tumors were harvested 1 h post radiation.

Tissue homogenates for pharmacodynamic analyses were collected and processed as follows. Tumor tissues from FADU or MDA-MB-231 tumors were flash frozen in liquid nitrogen and pulverized on dry ice using a tissue pulverizer. A portion of the tumor powder was transferred into a microtube, followed by addition of 500 μL of RIPA buffer [50 nM Tris, pH 8.0, 150 mM NaCl, 0.1% SDS, 0.5% sodium deoxycholate, 1% IGEPAL® CA-630 (Sigma, I8896)]. To 10 ml of RIPA buffer, 2 tablets each of Protease inhibitor and Phosphatase inhibitor (Roche, #04693159001 & #04906837001), 200 μL of 0.1 M PMSF and 160 μL of Aprotinin, (Sigma, A6279) were added. The suspension was homogenized on ice with a pellet pestle motor (KONTES) and incubated on ice for 30 minutes. Cell lysates were centrifuged at 13,200 rpm for 15 min at 4° C. and supernatants were transferred to a new tube and analyzed for protein concentration with a BCA kit (Pierce, 23227), followed by immunoblotting.

In vivo tumor growth delay study was performed as follows. Following implantation of the tumor cell lines, mice were monitored once weekly for tumor development. Upon development of a tumor measuring at least 100 mm³, tumor-bearing mice were stratified to one of 4 groups, utilizing 5 mice per group. Tumor volumes were between 100 and 500 mm³. Vehicle or compound 569 (10 mg/kg) were administered by oral gavage once daily for three consecutive days (qd×3), alone or in combination with focal radiation. The delivered volume of vehicle or compound 569 was adjusted based on body weight of the individual animals. Mice stratified to the radiation therapy groups received 3 Gy per day for three consecutive days (qd×3) 45 minutes following administration of the vehicle or compound 569. The mice were anesthetized with isoflurane during the radiation treatment, which was performed with the X-RAD 225Cx small animal image-guided irradiator (Precision X-Ray). The 40 mm×40 mm irradiation field was centered on the tumor-bearing leg via fluoroscopy. Mice were irradiated with parallel-opposed anterior and posterior fields of 225 kVp, 13 mA X-rays using a 0.3 mm Cu filter at an average dose rate of 300 cGy/min.
Group 1 mice received vehicle qd×3.
Group 2 mice received 10 mg/kg Compound 569 qd×3.
Group 3 mice received vehicle 45 minutes prior to 3 Gy focal irradiation qd×3.
Group 4 mice received 10 mg/kg Compound 569 45 minutes prior to 3 Gy focal irradiation qd×3.

### Statistical and Graphical Analysis of the In Vivo Data

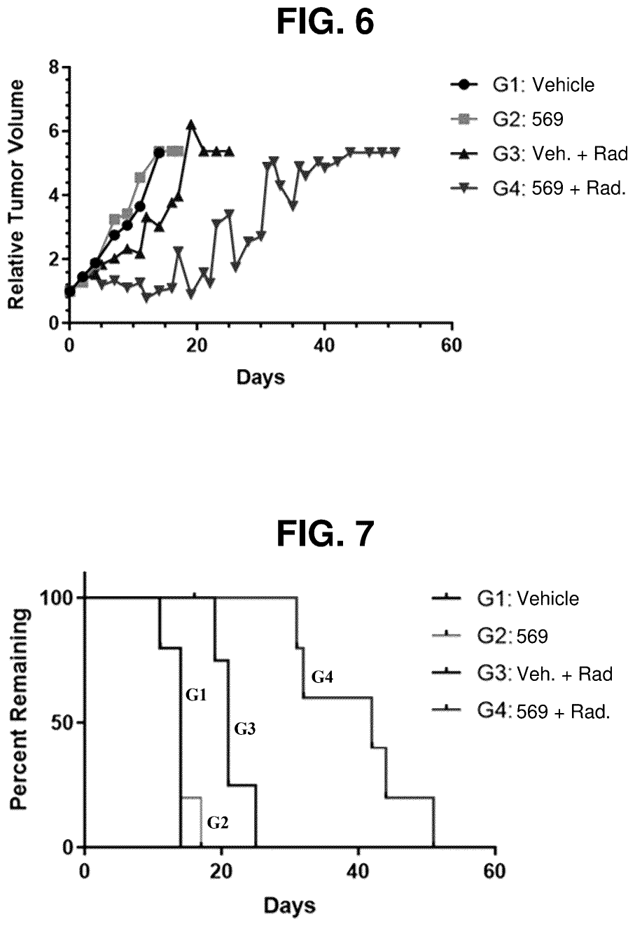

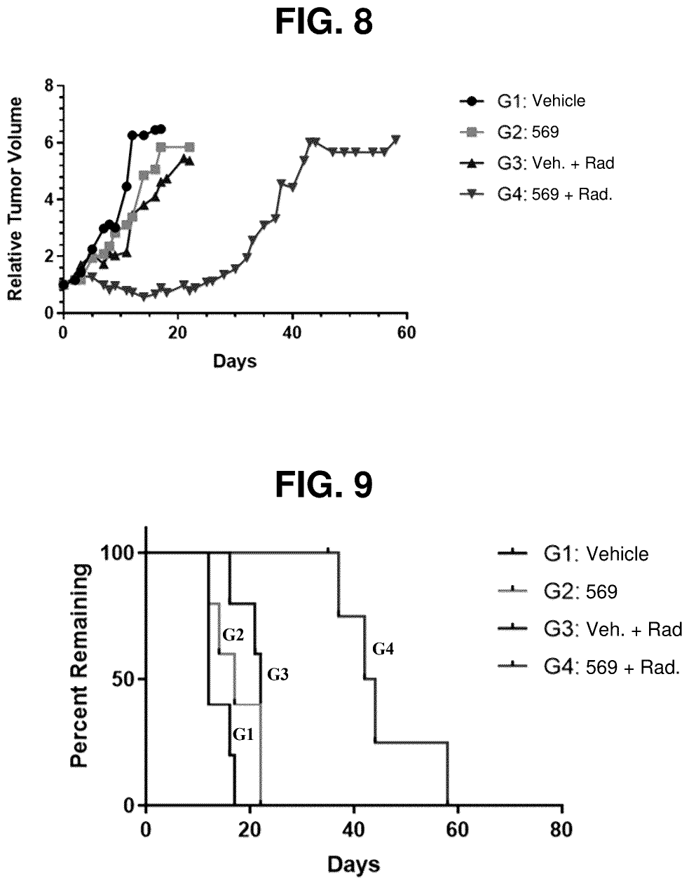

Prism (GraphPad) was used for graphical presentations. SAS 9.4 and R 3.6.1 were used for statistical analyses. Tumor growth rates among four treatment groups were compared. In order to adjust for the differing tumor sizes at the start of treatment, the relative tumor volume was calculated by dividing the tumor volume at each time point by the volume at baseline. Line plots were used to display median relative tumor volume over time (FIGS. 6 and 8). When an animal exited the study due to tumor quintupling, the final tumor volume recorded for the animal was included with the data used to calculate the median volume at subsequent time points. Kaplan-Meier plots were used to show the percentage of mice in each treatment group remaining in the study over time (FIGS. 7 and 9). Mice that were found dead prior to reaching the quintupling endpoint were subjected to right-censoring. Differences in quintupling-free survival between the four treatment groups were evaluated with a log-rank test. A P-value less than 0.05 was considered statistically significant.

### Results

FIG. 1 shows an image of an immunoblot from an in vitro experiment assessing the effect of compound 569 on MCF7 cells with or without radiation. The immunoblot shows the inhibition of radiation-induced autophosphorylation of ATM and DNA-PK kinases and radiation-induced phosphorylation of KAP1, an ATM substrate, by compound 569 in tumor cells. MCF7, a human breast carcinoma cell line, was pre-treated for 30 min with compound 569 at concentrations of 20, 100, 500, and 1000 nM. DMSO was used as a negative control. The cells were then exposed to 0 or 10 Gy of ionizing radiation (IR) and one hour later, cells were harvested for immunoblot analyses. Compound 569 showed potent dose-dependent inhibition of radiation-induced phosphorylation of DNA-PKcs at Ser2056 and ATM at Ser1981 (both autophosphorylation events) and the ATM substrate KAP1 (ser824) demonstrating compound 569 inhibited both DNA-PK and ATM kinases in a cell.

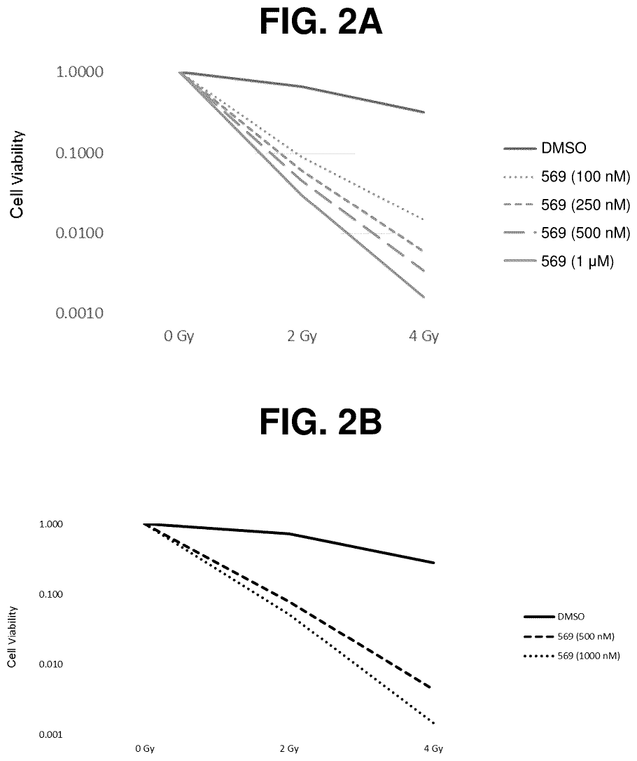

FIGS. 2A and 2B demonstrates the radiosensitizing properties of compound 569 in a clonogenic survival assay in vitro. MCF7 cells were pre-treated with vehicle or compound 569 at 100, 250, 500, and 1000 nM for 30 min before irradiation with increasing doses of IR (0, 2, and 4 Gy). After 5 h, the medium was removed and fresh medium without inhibitors was added to cells, and they were cultured for 9 days before being fixed, stained and colonies counted. The 5-h exposure of cells to compound 569 induced a significant degree of radiosensitization without evidence of cellular toxicity in the absence of IR in MCF7 cells (FIG. 2A, Table 4A).

FIG. 2A demonstrates the radiosensitizing properties of compound 569 in a clonogenic survival assay in vitro. A549 cells were pre-treated with vehicle or compound 569 at 500 and 1000 nM for 30 min before irradiation with increasing doses of IR (0, 2, and 4 Gy). After 5 h, the medium was removed and fresh medium without inhibitors was added to cells, and they were cultured for 9 days before being fixed, stained and colonies counted. The 5-h exposure of cells to compound 569 induced a significant degree of radiosensitization without evidence of cellular toxicity in the absence of IR in A549 cells (FIG. 2B, Table 4B).

FIG. 3 is an immunoblot showing the induction of phosphorylation of TBK1 by compound 569, AZD0156 (a selective ATM inhibitor; ATMi), peposertib (a selective DNA-PK inhibitor; DNA-PKi), and a combination of AZD0156 and peposertib (Ai+Di) in HCT116 cells expressing wild-type p53 or HCT116 cells that were negative for p53 expression. Phosphorylation of TBK1 is a marker of activation of the type I interferon response and has been linked to enhanced tumor response to immune checkpoint blockade therapy. Dual inhibition of ATM and DNA-PKcs by combining the two selective chemical inhibitors (AZD0156 and peposertib) together resulted in more profound activation of TBK1 than either of the selective inhibitors alone. Similarly, exposure of cells to compound 569, a dual inhibitor of ATM and DNA-PKcs, was a more potent activator of TBK1 phosphorylation than either of the single target kinase inhibitors and was independent of p53 status. The TBK1 activation by Compound 569 and dual inhibition of ATM and DNA-PKcs appeared to be independent of the cGAS and phospho-STING induction.

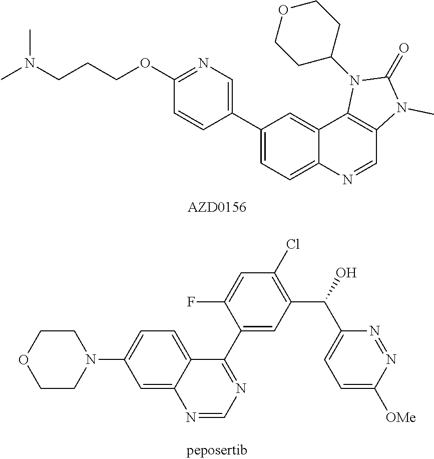

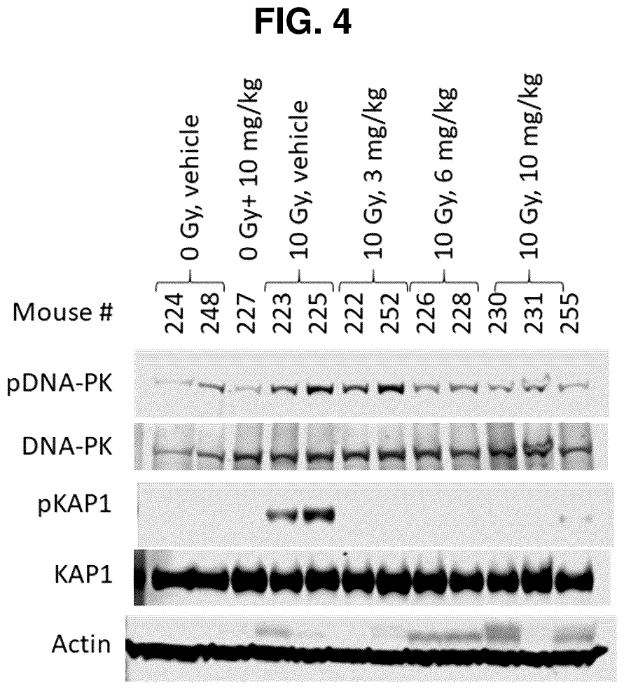

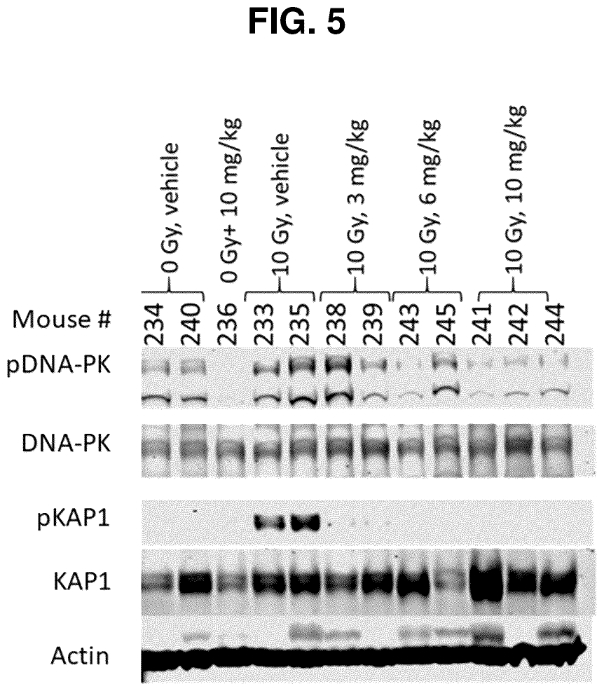

To assess the pharmacodynamic activity of Compound 569 in vivo, FADU and MDA-MB-231 cells were grown as tumor xenografts in nude mice. Vehicle or compound 569 were administered by oral gavage alone or in combination with focal radiation. Mice were dosed with vehicle or compound 569 at 3, 6, and 10 mg/kg. Tumors then received 10 Gy of focal irradiation 45 min later or were left unirradiated (0 Gy, vehicle and 0 Gy+compound 569 at 10 mg/kg). Tumors were harvested 1 h post radiation. Immunoblotting of lysates from these tumors shows radiation induced autophosphorylation of pDNA-PK and radiation-induced phosphorylation of KAP1, a downstream target of ATM and dose-dependent inhibition of those phosphorylation events in FADU and MDA231 tumors treated with radiation plus compound 569. Tumor levels of radiation-induced autophosphorylation of DNA-PK and radiation-induced phosphorylation of KAP1 were significantly inhibited by ˜55% and >80%, respectively at 10 mg/kg (FIGS. 4 and 5, Tables 6-8). Basal phosphorylation of DNA-PK was also inhibited by compound 569 in tumors.

TABLE 4A
Compound 569 Clonogenic Assay in MCF7 cells (see FIG. 2A)

| | Clonogenic | Clonogenic | | Clonogenic | | % survival @ |
|---|---|---|---|---|---|---|
| | survival | survival | | survival | | 0 Gy w/out |
| Compound | ratio | ratio | Fold | ratio | Fold | IR |
| (concentration) | 0 Gy | 2 Gy | 2 Gy | 4 Gy | 4 Gy | 100% |
| DMSO | 1.000 | 0.662 | 1.0 | 0.317 | 1.0 | 97% |
| 569 (100 nM) | 1.000 | 0.0882 | 7.5 | 0.0146 | 21.8 | 92% |
| 569 (250 nM) | 1.000 | 0.0610 | 10.8 | 0.00588 | 54.0 | 97% |
| 569 (500 nM) | 1.000 | 0.0451 | 14.7 | 0.00347 | 91.5 | 105% |
| 569 (1000 nM) | 1.000 | 0.0301 | 22.0 | 0.00160 | 198 | 100% |

TABLE 4B
Compound 569 Clonogenic Assay in A549 cells (see FIG. 2A)

| | Clonogenic | Clonogenic | | Clonogenic | | |
|---|---|---|---|---|---|---|
| | survival | survival | | survival | | % survival @ |
| Compound | ratio | ratio | Fold | ratio | Fold | 0 Gy w/out |
| (concentration) | 0 Gy | 2 Gy | 2 Gy | 4 Gy | 4 Gy | IR |
| DMSO | 1.000 | 0.7272 | 1.0 | 0.2818 | 1.0 | 100% |
| 569 (500 nM) | 1.000 | 0.0780 | 9.3 | 0.0044 | 64 | 110% |
| 569 (1000 nM) | 1.000 | 0.0520 | 14 | 0.0015 | 188 | 103% |

TABLE 5
Quantification of pDNA-PK/DNA-PK in FADU tumors

| | pDNA- | Standard |
|---|---|---|
| Treatment | PK/DNA-PK | deviation |
| No radiation, vehicle | 1.14 | 0.263 |
| No radiation, 10 mg/kg | 0.343 | NA |
| compound 569 | | |
| 10 Gy, vehicle | 1 | 0.0565 |
| 10 Gy, 3 mg/kg compound 569 | 0.938 | 0.181 |
| 10 Gy, 6 mg/kg compound 569 | 0.658 | 0.126 |
| 10 Gy, 10 mg/kg compound 569 | 0.476 | 0.188 |

TABLE 6
Quantification of pKAP1/KAP1 in FADU tumors

| | | Standard |
|---|---|---|
| Treatment | pKAP1/KAP1 | deviation |
| No radiation, vehicle | 0.00505 | 0.000265 |
| No radiation, 10 mg/kg | 0.00595 | NA |
| compound 569 | | |
| 10 Gy, vehicle | 1 | 0.0616 |
| 10 Gy, 3 mg/kg compound 569 | 0.0903 | 0.005 |
| 10 Gy, 6 mg/kg compound 569 | 0.0982 | 0.0741 |
| 10 Gy, 10 mg/kg compound 569 | 0.156 | 0.171 |

TABLE 7
Quantification of pDNA-PK/DNA-PK in MDA-231 tumors

| | pDNA- | Standard |
|---|---|---|
| Treatment | PK/DNA-PK | deviation |
| No radiation, vehicle | 0.648 | 0.0948 |
| No radiation, 10 mg/kg | 0.223 | NA |
| compound 569 | | |
| 10 Gy, vehicle | 1 | 0.231 |
| 10 Gy, 3 mg/kg compound 569 | 0.889 | 0.414 |
| 10 Gy, 6 mg/kg compound 569 | 0.670 | 0.278 |
| 10 Gy, 10 mg/kg compound 569 | 0.454 | 0.116 |

TABLE 8
Quantification of pKAP1/KAP1 in MDA-231 tumors

| | | Standard |
|---|---|---|
| Treatment | pKAP1/KAP1 | deviation |
| No radiation, vehicle | 0.00357 | 0.000826 |
| No radiation, 10 mg/kg | −0.00319 | NA |
| compound 569 | | |
| 10 Gy, vehicle | 1 | 0.114 |
| 10 Gy, 3 mg/kg compound 569 | 0.327 | 0.158 |
| 10 Gy, 6 mg/kg compound 569 | 0.0130 | 0.00879 |
| 10 Gy, 10 mg/kg compound 569 | 0.00901 | 0.00242 |

### Effects on FADU Tumors

Mice in Group 1 received vehicle, qd×3. Aggregate tumor growth in this group was progressive. The median time to endpoint was 14 days with a range of 11-14 days (FIGS. 6 and 7). Mice in Group 2 received Compound 569 at a dose of 10 mg/kg, qd×3. The median time to endpoint was 14 days with a range of 11 to 17 days. The tumor growth curves and quintupling-free survival for Group 2 animals are nearly identical to those for Group 1 (FIGS. 6 and 7). Mice in Group 3 received vehicle and a focal radiation dose of 3 Gy, which was delivered 45 minutes after administration of the vehicle. Both the vehicle and radiation were qd×3. One animal was found dead on Day 16 due to unknown causes. The median time to endpoint was 21 days with a range of 19 to 25 days. Overall survival was significantly improved compared to Group 1 and Group 2 (P<0.01, logrank). The delay in aggregate tumor growth and increase in quintupling-free survival for Group 3 relative to Groups 1 and 2 is shown in FIGS. 6 and 7. Mice in Group 4 received compound 569 and a focal radiation dose of 3 Gy, which was delivered 45 minutes after administration of the compound. Both compound 569 and radiation were administered qd×3. The median time to endpoint was 42 days with a range of 31 to 51 days. Overall survival was significantly improved compared to Group 1, Group 2, and Group 3 (P<0.01, logrank). The delay in aggregate tumor growth and increase in quintupling-free survival for Group 4 relative to all other groups is shown in FIGS. 6 and 7. All treatments were well-tolerated.

### Effects on MDA-MB-231 Tumors

Mice in Group 1 received vehicle, qd×3. The median time to endpoint was 12 days with a range of 12 to 17 days. Mice in Group 2 received compound 569 at a dose of 10 mg/kg, qd×3. The median time to endpoint was 17 days with a range of 12 to 22 days. The tumor growth curves and quintupling-free survival for Group 2 animals are nearly identical to those for Group 1 (FIGS. 8 and 9). Mice in Group 3 received vehicle and a focal radiation dose of 3 Gy, which was delivered 45 minutes after administration of the vehicle. Both the vehicle and radiation were qd×3. The median time to endpoint was 22 days with a range of 16 to 22 days. Overall survival was significantly improved compared to Group 1 animals treated with vehicle alone (P<0.01, logrank). The increase in quintupling-free survival for Group 3 relative to Groups 1 and 2 is shown in FIG. 9. Mice in Group 4 received 10 mg/kg compound 569 and a focal radiation dose of 3 Gy, which was delivered 45 minutes after administration of compound. Both compound 569 and radiation were qd×3. One animal was found dead on Day 35 due to unknown causes. The median time to endpoint was 43 days with a range of 37 to 58 days. Overall survival was significantly improved compared to Group 1, Group 2, and Group 3 (P<0.01, logrank). The delay in aggregate tumor growth and increase in quintupling-free survival for Group 4 relative to all other groups is shown in FIGS. 8 and 9. All treatments were well-tolerated.

### Example 4. Tolerability Study for Compound 569 in Murine Models

A murine model is to be used to test the tolerability of Compound 569 according to the following procedure:

Mice are divided into two groups, one to be treated with 9 mg/kg of Compound 569 (Group 1; mesylate salt), and one to be treated with 10 mg/kg of Compound 569 (Group 2; mesylate salt). The groups and treatment conditions are provided in Table 9 below:

TABLE 9
Treatment Conditions

| Group | | Animals/ | Dose | Vol. | | | |
|---|---|---|---|---|---|---|---|
| # | Agent | group | (mg/kg) | (ml/kg) | Route | Regimen | Duration |
| 1 | Compound | 5 | 10 | 10 | po | BID day | 21 days |
| | 569 | | | | | 1, 3, 5, | |
| | | | | | | 7, 9, 11, | |
| 2 | Compound | 5 | 9 | 10 | po | BID day | 19 days |
| | 569 | | | | | 1-5, 8-12, | |
| | | | | | | 15-19 | |

Note: BID interval 12 h

Vehicle for Compound 569 is 0.5% HPMC and 0.1% Tween 80 in deionized water. Compound 569 is used as the mesylate salt, prepared daily. Dosing volume is 10 ml/kg, adjusted for bodyweight. For routine monitoring, all study animals are monitored for behavior such as mobility, food and water consumption (by cage side checking only), body weight (BW), eye/hair matting and any other abnormal effect. Any mortality and/or abnormal clinical signs are recorded daily and communicated. Body weights of all animals are measured daily throughout the study and recorded.

### Example 5. Study to Determine Efficacy of Compound 569 Alone and in Combination with Anti-PD1 Antibody in Murine Models

This example investigates anti-tumor efficacy of Compound 569 (as an exemplary Formula (I) compound; mesylate salt) in murine models, either taken alone or in combination with an immune checkpoint inhibitor (using an anti-PD1 antibody as an example).

C57BL/6 mice between 8 and 12 weeks old are injected with 5×10⁵ MC38 tumor cells in 0.1 mi/mouse of 0% Matrigel sc in the flank. Mice are paired based on tumor size once the tumors reach approximately 80-120 mm³. Randomization occurs on day 1 and dosing begins on day 2 in order to support BID dosing schedule. Body weight is measured daily for 6 days, then biweekly until the end of the study. Tumor size is measured by caliper biweekly throughout the study. Animals are monitored individually until the tumor volume reaches 1500 mm³ or 45 days, whichever comes first, though responding animals may be measured longer.

Dosage regimens for each mice group are selected as shown in Table 10. On days where Compound 569 is administered with anti-PD1 antibody, Compounds 569 is given first. Doses are prepared from the mesylate salt form of compound 569 dissolved in aqueous solution with 0.5% hydroxypropyl methylcellulose and 0.1% Tween 80 in deionized water. Anti-PD1 RMP1-14 antibody doses are prepared in phosphate buffered saline. Clone of RMP1-14 is RMP1-14 ICHOR SKU: IHC1132. Rat IgG2a is prepared in phosphate buffered saline. Clone of Rat IgG2a is 2A3 Rat IgG2a Isotype control: BioXcell cat #BE0089.

TABLE 10
Combination Therapy Treatment Conditions

| Gr. | N | Agent | Active dose | Route | Schedule |
|---|---|---|---|---|---|
| 1^{#} | 10 | Rat IgG2a | 5 mg/kg | ip | biwk × 2 (start on day 2 - |
| | | | | | Days 2, 5, 9, 12) |
| 2 | 10 | anti-PD-1 | 5 mg/kg | ip | biwk × 2 (start on day 2 - |
| | | RMP1-14 | | | Days 2, 5, 9, 12) |
| 3 | 10 | XRD-0394 | 9 mg/kg | po | bid days 2-6, 9-13, 16 |
| 4 | 10 | XRD-0394 | 10 mg/kg | po | bid days 2, 4, 6, 8, |
| | | | | | 10, 12, 14, 16 |
| 5 | 10 | anti-PD-1 | 5 mg/kg// | ip//po | biwk × 2 (start on day 2 - |
| | | RMP1-14// | 9 mg/kg | | Days 2, 5, 9, 12)//bid |
| | | XRD-0394 | | | days 2-6, 9-13, 16 |
| 6 | 10 | anti-PD-1 | 5 mg/kg// | ip//po | biwk × 2 (start on day 2 - |
| | | RMP1-14// | 10 mg/kg | | Days 2, 5, 9, 12)//bid |
| | | XRD-0394 | | | days 2, 4, 6, 8, |
| | | | | | 10, 12, 14, 16 |

Note:
BID interval 12 h.

### Example 6. Study to Determine Efficacy of Compound 569 in Combination with Anti-PD1 Antibody, Radiotherapy, or Both in MC38 Murine Models

This study investigates the anti-tumor effects of compound 569 in combination with an anti-PD1 antibody and/or radiotherapy.

### Methods

### Syngeneic Murine Tumor Model

The murine colon adenocarcinoma cell line, MC38-NCI.TD1 (MC38), was cultured in Dulbecco's modified Eagle's medium (DMEM), 10% fetal bovine serum (FBS), 25 μg/ml gentamicin, 2 mM L-glutamine, 1% penicillin/streptomycin/L-glutamine at 37° C. in 5% CO2. Cells were dissociated in 0.25% trypsin/2.21 mM ethylenediaminetetraacetic acid in Hank's Balanced Salt Solution and brought to a density of 5×10⁶ cells/200 μL in DMEM. Tumors were initiated by injection of the cell suspension subcutaneously in the right axilla of female C57BL/6 mice (Jackson Laboratory). All mice were sorted into 7 study groups (n=10) based on caliper estimation of tumor burden. Mice were distributed to ensure that the mean tumor burden for all groups was within 10% of the overall mean tumor burden for the study population (90 mm³). Mice were monitored daily for clinical signs and weighed and measured three times weekly. The study endpoint was 30 days post staging or when tumor burden was >2000 mm³.

### Formulation and Administration

The vehicle for compound 569 was 0.5% hydroxypropyl methyl cellulose (HPMC) and 0.1% Tween 80 dissolved in deionized water. Vehicle for compound 569 and compound 569 (12 mg/kg) were administered by oral gavage once daily starting the day of staging for two consecutive days (qd×2), in combination with focal radiation. Mice stratified to the radiation therapy groups received 5 Gy per day for two consecutive days (qd×2) 60 minutes following administration of the vehicle or compound 569. The mice were anesthetized with isoflurane during the radiation treatment, which was performed with the Xstrahl Life Sciences Small Animal Radiation Research Platform (Xstrahl, Inc.) Mice were irradiated at an average dose rate of 2.5 Gy/min.

The vehicle for isotype control (clone 2A3) anti-mouse PD-1 (anti-mPD-1; Bio X Cell) and anti-mPD-1 (Clone RMP1-14; Bio X Cell) was phosphate buffered saline (PBS). Isotype control anti-mPD-1 (10 mg/kg) and anti-mPD-1 (10 mg/kg) were administered intraperitonealy twice weekly for 2 weeks starting day 2 post staging. Treatment schedules for the various groups of animals are summarized below:
Group 1 mice received vehicle qd×2+10 mg/kg isotype control (q3d×2, 3 days off)×2; start 2 days post stage.
Group 2 mice received 10 mg/kg anti-mPD-1 (q3d×2, 3 days off)×2; start 2 days post stage.
Group 3 mice received focal irradiation 5 Gy qd×2+10 mg/kg isotype control (q3d×2, 3 days off)×2; start 2 days post stage.
Group 4 mice received focal irradiation 5 Gy qd×2+10 mg/kg anti-mPD-1 (q3d×2, 3 days off)×2; start 2 days post stage.
Group 5 mice received 12 mg/kg Compound 569 qd×2 (1 hr prior to focal irradiation)+5 Gy qd×2
Group 6 mice received 12 mg/kg Compound 569 qd×2+10 mg/kg anti-mPD-1 (q3d×2, 3 days off)×2; start 2 days post stage.
Group 7 mice received 12 mg/kg Compound 569 qd×2 (1 hr prior to focal irradiation)+5 Gy qd×2+10 mg/kg anti-mPD-1 (q3d×2, 3 days off)×2; start 2 days post stage.

### Data Analysis

ΔC and ΔT—Are individual mouse endpoints that are calculated for each mouse as follows:

$$Δ T = {T}_{t} - {T}_{0} and Δ C = {C}_{t} - {C}_{0} ,$$

Where Tₜ and T₀ are the tumor burdens of a treated mouse at time t or at the initiation of dosing, respectively. ΔC reflects similar calculations for the control mice.
Median ΔT/ΔC—Is a group endpoint. It is calculated for each day of treatment as:

$$Median Δ T / Δ C = ( Δ {T}_{med} / Δ {C}_{med} ) * 100 = ( median ( {T}_{t} / {T}_{0} ) / median ( {C}_{t} / {C}_{0} ) ) * 100$$

The results are presented as a %. When the median ΔT/ΔC is negative (the median treated tumor burden is regressing), the median ΔT/ΔC is not reported and the Median % Regression is reported instead.
% Regression—Is a group endpoint. It indicates the percentage reduction in the Median tumor volume from baseline. It is calculated as:

$$\% Regression = - ( Δ {T}_{med} / {T}_{0 med} ) * 100$$

Day 20 post staging was chosen for ΔT/ΔC since it was the last day all animals were on study prior to euthanasia due to tumor burden.
Complete Regression (CR)—An animal is credited a complete regression if its tumor burden is reduced to an immeasurable volume at any point after the first treatment. The CR must be maintained for at least 2 consecutive measurements.
Tumor-Free Survivor (TFS)—A TFS is any animal that survives until termination of the study, and has no reliably measurable evidence of disease at study termination.

Statistical analysis was performed using 1-way ANOVA followed by Dunn's Multiple Comparison Test.

### Results

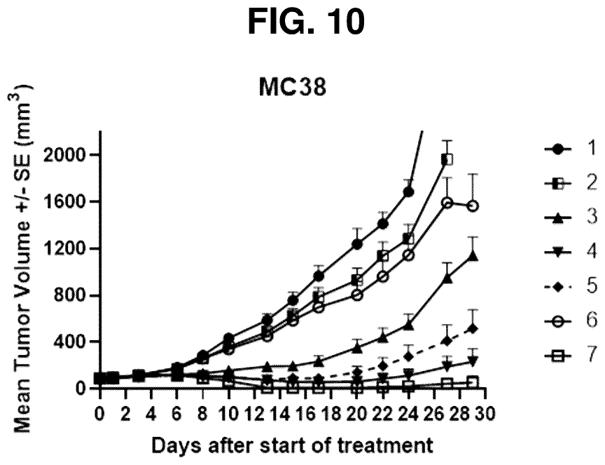

Treatment with vehicle in combination with isotype control (Group 1) was used as the overall study control. Treatment with anti-mPD-1 as a single agent (Group 2) was only slightly better than control, resulting in a Day 20 median ΔT/ΔC value of 83% (p>0.05). See FIG. 10. The addition of compound 569 in combination with anti-mPD-1 (Group 6) showed limited increase in efficacy compared to single agent anti-mPD-1 treatment (Day 20 median ΔT/ΔC value of 64%, p>0.05) and the combination of compound 569 and the anti-mPD-1 antibody showed increased efficacy as compared with the control group.

Treatment with radiation in combination with isotype control (Group 3) produced anti-cancer activity with a Day 20 median ΔT/ΔC value of 20% (p>0.05), but did not result in significant tumor regressions.

Mice in groups with dual radiation combination treatments exhibited varying degrees of anti-tumor activity. A significant enhancement in anti-tumor activity was seen with the addition of anti-mPD-1 (group 4) or 569 (Group 5) in combination with radiation, resulting in Day 20 regression values of 64% (p<0.05) and 22% (p<0.05), respectively. Treatment in Group 4 resulted in a 50% incidence of complete regressions (CR) and tumor free survivors (TFS). Treatment in Group 5 resulted in a 50% incidence of CR and a 30% incidence of TFS. Triple combination treatment with anti-mPD1, 569 and radiation (Group 7) produced the most substantial anti-tumor activity, demonstrating an increase in efficacy compared to all other single or dual agent treatments. Treatment resulted in a Day 20 regression value of 99% (p<0.05). Mice in Group 7 also exhibited a 90% incidence of CR and TFS.

All treatments resulted in minimal body weight loss. Both the control group (Group 1) and the compound 569 and anti-mPD-1 combination treatment group (Group 6) exhibited 1 death (10% incidence).

In sum, the combination of compound 569, anti-PD1 antibody, and radiotherapy showed superior anti-tumor activity in the animal model as compared with compound 569 in combination with either the anti-PD1 antibody or radiotherapy.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for treating cancer, comprising administering to a subject in need thereof an effective amount of a compound of Formula (I): 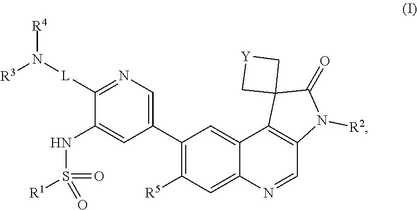
or a pharmaceutically acceptable salt thereof;
wherein:
R⁵ is H or halogen;
Y is CHR⁶ or NR⁷;
L is —OR⁸—, or —N(R⁸)₂—, wherein each R⁸ is independently H or C₁₋₃ alkyl, or both R⁸ substituents, together with N, form a heterocyclyl ring; and
R¹, R², R³, R⁴, R⁶, and R⁷ are each independently H, or C₁₋₃ alkyl; and
wherein the subject has received or is receiving an anti-tumor immune checkpoint inhibitor and a radiotherapy.

2. A method for treating cancer, comprising administering to a subject in need thereof (a) an effective amount of an anti-tumor immune checkpoint inhibitor and a radiotherapy, and (b) an effective amount of a compound of Formula (I) or a pharmaceutically acceptable salt thereof: 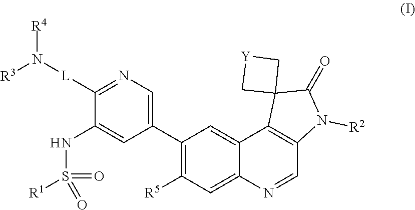
wherein:
R⁵ is H or halogen;
Y is CHR⁶ or NR⁷;
L is —OR⁸—, or —N(R⁸)₂—, wherein each R⁸ is independently H or C₁₋₃ alkyl, or both R⁸ substituents, together with N, form a heterocyclyl ring; and
R¹, R², R³, R⁴, R⁶, and R⁷ are each independently H, or C₁₋₃ alkyl.

3. (canceled)

4. (canceled)

5. (canceled)

6. (canceled)

7. The method of claim 1, wherein the compound is of Formula (Ia): 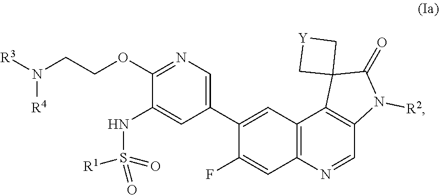
or a pharmaceutically acceptable salt thereof, wherein R¹-R⁴ are as defined in claim 1.

8. The method of claim 7, wherein the compound is of Formula (Ia-1): 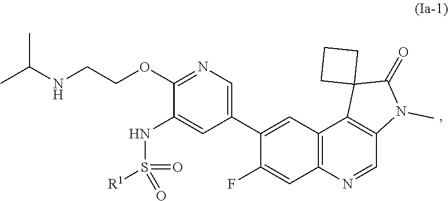
or a pharmaceutically acceptable salt thereof, wherein R¹ is C₁₋₃ alkyl.

9. The method of claim 8, wherein the compound of Formula (Ia-1) is 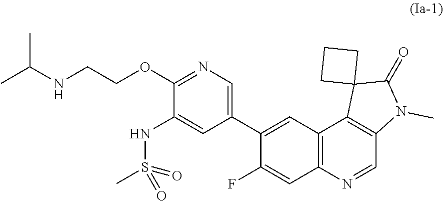 [SMILES: CC(C)NCCOc1ncc(-c2cc3c4c(cnc3cc2F)N(C)C(=O)C42CCC2)cc1NS(C)(=O)=O]
or a pharmaceutically acceptable salt thereof.

10. The method of claim 1, wherein the compound is of Formula (Ib): 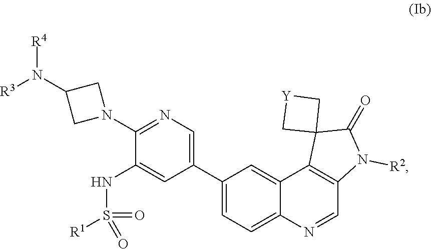
or a pharmaceutically acceptable salt thereof, wherein R¹-R⁴ are as defined in claim 1.

11. (canceled)

12. The method of claim 1, wherein the compound of Formula (I) is administered orally once or twice a day.

13. (canceled)

14. The method of claim 1, wherein the checkpoint inhibitor is a PD-1 antagonist, which optionally is selected from the PD-1 antagonists listed in Table 1.

15. The method of claim 14, wherein the PD-1 antagonist is an anti-PD-1 antibody, which optionally is selected from the anti-PD-1 antibodies listed in Table 1.

16. The method of claim 15, wherein the PD-1 antagonist is an anti-PD-L1 antibody, which optionally is selected from the anti-PD-L1 antibodies listed in Table 1.

17. The method of claim 1, wherein the checkpoint inhibitor is administered by intravenous infusion or orally.

18. The method of claim 1, wherein the subject is a human cancer patient.

19. The method of claim 16, wherein the human cancer patient has colon cancer, melanoma, breast cancer, lung cancer or head and neck cancer.

20. A mesylate salt of a compound of Formula (I): 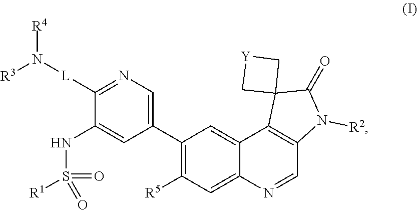
wherein:
R⁵ is H or halogen;
Y is CHR⁶ or NR⁷;
L is —OR⁸—, or —N(R⁸)₂—, wherein each R⁸ is independently H or C₁₋₃ alkyl, or both R⁸ substituents, together with N, form a heterocyclyl ring; and
R¹, R², R³, R⁴, R⁶, and R⁷ are each independently H, or C₁₋₃ alkyl.

21. The mesylate salt of claim 20, wherein the compound is of Formula (Ia): 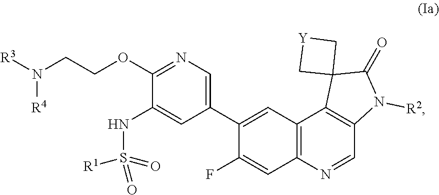
wherein R¹-R⁴ are as defined in claim 20.

22. The mesylate salt of claim 21, wherein the compound is of Formula (Ia-1): 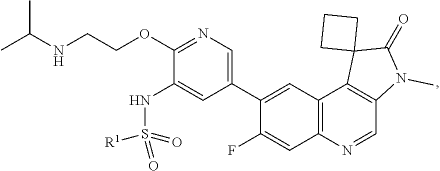
and wherein R¹ is C₁₋₃ alkyl.

23. The mesylate salt of claim 22, which has the following structure:

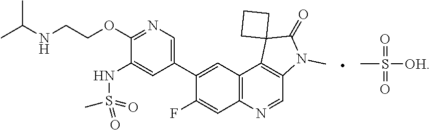

24. The mesylate salt of claim 20, wherein the compound is of Formula (Ib): 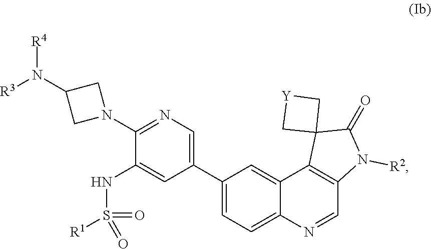
wherein R¹-R⁴ are each independently H, or C₁₋₃ alkyl.

25. A pharmaceutical composition, comprising a mesylate salt of claim 20.

26. A method for treating cancer, comprising administering to a subject in need thereof an effective amount of the mesylate salt of the compound of Formula (I) of claim 20.
